(19) United States
(12) Patent Application Publication
(10) Pub. No.: US 2012/0077960 A1
(43) Pub. Date: Mar. 29, 2012

(54) FLUOROBODIES: INTRINSICALLY FLUORESCENT BINDING LIGANDS

(75) Inventors: Andrew M. Bradbury, Sante Fe, NM (US); Geoffrey S. Waldo, Sante Fe, NM (US); Csaba Kiss, Los Alamos, NM (US); Devin Close, Los Alamos, NM (US)

(73) Assignee: Los Alamos National Security, LLC, Los Alamos, NM (US)

(21) Appl. No.: 13/256,198

(22) PCT Filed: Mar. 12, 2010
(86) PCT No.: PCT/US10/00751

§ 371 Date: Dec. 8, 2011

Related U.S. Application Data
(60) Provisional application No. 61/210,084, filed on Mar. 13, 2009.

(51) Int. Cl. C07K 14/00; C07H 21/04

(52) U.S. Cl. 530/350; 536/23.1

(57) ABSTRACT

Binding ligands with intrinsic fluorescence (“fluorobodies”), fluorobody libraries, and methods of preparing fluorobodies are provided. In one aspect, the invention provides fluorobodies generated from a highly stable, artificial fluorescent protein, eCGP123 and derivatives thereof.

### STATEMENT AS TO RIGHTS TO INVENTIONS MADE UNDER FEDERALLY SPONSORED RESEARCH OR DEVELOPMENT

This invention was made with government support under Contract No. DE-AC52-06 NA 25396 awarded by the U.S. Department of Energy. The government has certain rights in the invention.

### BACKGROUND OF THE INVENTION

Molecular diversity libraries with billions of different members have proved to be rich sources of binding ligands. Although peptides ^{1,2} and antibodies ^{3,4,5} have been most commonly used, other ligands, such as CTLA4 ⁶, lipocalins ⁷, protein A ⁸, isolated light ⁹ or heavy chain ¹⁰ variable regions have also been developed.

In general, binding ligands have attempted to recapitulate the binding of antibodies, with regions of diversity (binding elements) concentrated on one face of the protein. However, none of these ligands have any function beyond binding. Moreover, subsequent detection always requires the use of tags or secondary binding reagents. A binding ligand which had intrinsic detection capability, such as fluorescence, would have enormous potential, providing a real time indication of binding as well as ligand functionality and concentration.

Green fluorescent protein (GFP) from the luminescent jellyfish Aequorea victoria is an intrinsically fluorescent protein ¹¹ which is now in widespread use as a detection agent in numerous applied contexts. However, although GFP has been displayed on the surface of bacteria ¹², no GFP based libraries have been created or used in binding selection experiments. Attempts to insert linkers or random peptides within GFP^{13,14} have on the whole been unsuccessful, with most insertions rendering the GFP either non- or weakly fluorescent, presumably due to deleterious effects on folding. One report has described the identification of GFP loop inserted peptide sequences with apparent nuclear localization activity ¹⁴, but at very high cytoplasmic GFP concentrations.

A report in 2003 appeared to describe the development of “fluorobody” affinity reagents based on the insertion of up to four HCDR3 into sfGFP¹⁵. However, this report was subsequently retracted¹⁶, and it was reported that the fluorescence of clones containing a single insert was extremely low, and clones containing three or four inserts were completely non-fluorescent.

The one success in the transformation of a fluorescent protein into a specific fluorescent binder¹⁷ involved the insertion of a library of antibody binding loops derived from the human heavy chain third complementarity determining region (HCDR3) from a pool of human donor lymphocytes inserted into a single site of superfolder GFP (sfGFP), a particularly stable form evolved for improved folding¹⁸, using a display platform based on T7 phage. However, no subsequent report of actual binders emerged.

Other reports describe the use of GFP as a potential optical signaling protein, with GFP fluorescence (or FRET) modulated by changes in voltage ¹⁹, β-lactamase inhibitory protein concentration ²⁰, calcium ions ²¹, zinc ions ²¹ or pH²². Furthermore, the potential of fluorescent GFP constructs containing insertions designed to measure changes in phosphorylation, protease activity, glutamate concentration, and redox state have been described, although not reduced to practice ²¹.

In general, the environmental modification of GFP fluorescence is mediated by the insertion of additional protein domains within the GFP sequence, with all but one ²⁰ of such modified GFPs having insertions at a single position: either tyrosine 145, or the equivalent of tyrosine 145 after circular permutation.

A large number of other fluorescent and chromophoric proteins related to GFP isolated from other luminescent and/or chromophoric organisms have now been described (see Zimmer, 2002, Chem. Rev. 102: 759-781; Verkhusha et al., 2003, GFP-like fluorescent proteins and chromoproteins of the class Anthozoa. In: Protein Structures: Kaleidescope of Structural Properties and Functions, Ch. 18, pp. 405-439, Research Signpost, Kerala, India) Additionally, various mutants of these fluorescent proteins have been created in order to provide enhanced or altered biological properties. With few exceptions, all of the known fluorescent proteins maintain a characteristic 11-stranded beta-barrel three dimensional structure which surrounds a centrally-located self-activating chromophore. As a group, the fluorescent proteins display a broad range of excitation and emission spectra, characteristics which may be shifted by mutation.

More recently, consensus green protein (CGP), a completely synthetic fluorescent protein was described²³. The sequence of this protein was derived using an approach termed guided consensus engineering and was based on a comparison of 31 natural fluorescent protein sequences with a homology greater than 62% to monomeric Azami green²⁴. CGP was created by using the consensus amino acid, except where no consensus could be defined, in which case the monomeric Azami green amino acid was used. CGP was very fluorescent, but not particularly stable. In particular, insertion of potential binding loops into CGP resulted in loss of fluorescence.

Current methods to detect targets using binding ligands, e.g., antibodies, require the use of secondary detectors, such as secondary antibodies labeled with a detection moiety. The current invention provides binding ligands, such as GFP-based binding ligands, with intrinsic fluorescence or color. Thus, these ligands offer advantages over existing technologies as they do not require the use of other reagents either coupled to the protein or added to the reaction mixture to detect binding. For example, the fluorescent binding ligands of the invention, also referred to herein as “fluorobodies”, can be used to directly detect antigen binding in real time. In addition to being useful in all applications for which antibodies, or antibody fragments, are currently used (e.g., immunofluorescence, immunohistochemistry, immunoprecipitation, western blotting, ELISAs, inhibition assays and protein-protein interaction studies), fluorobodies can also be used in novel applications for which antibodies or antibody fragments are less suitable. Such applications include protein arrays, high throughput drug screening and biosensors.

### SUMMARY OF THE INVENTION

The invention provides binding ligands with intrinsic fluorescence (“fluorobodies”), fluorobody libraries, and methods of preparing fluorobodies. In one aspect, the invention provides fluorobodies generated from a highly stable, artificial fluorescent protein, eCGP123 and derivatives thereof. In another aspect, improved non-agregating variants of eCGP123 and fluorobodies generated therefrom are provided. In one embodiment, eCGP123-based fluorobodies comprise heterologous polypeptides to generate binding sites in at least two loop positions on the surface of the fluorescent protein. In a specific embodiment, described by way of example herein, a fluorobody comprising heterologous binding sites in three loop positions is provided.

The binding sites of a fluorobody of the invention may comprise random peptides, or a restricted set of random peptides (e.g. tyrosine, serine glycine), or may comprise complementarity determining regions (CDRs), such as human immunoglobulin CDR3s.

In another aspect, the invention provides an expression vector comprising a nucleic acid sequence encoding a fluorobody as set forth above, and additionally provides a host cell comprising the expression vector.

The invention also provides a library comprising a population of nucleic acid sequences encoding fluorobodies as set forth above. In some embodiments, the library comprises a nucleic acid sequence encoding a fluorobody that is linked to a polypeptide selected from the group consisting of a phage coat polypeptide, a bacterial outer membrane protein, a yeast external protein and a DNA binding protein. The library may be any kind of library, for example a display library such as a phage display library, a ribosomal display library, an mRNA display library, a bacterial display library, or a yeast display library.

In another aspect, the invention provides a method of preparing a fluorobody that binds to a target antigen, the method comprising inserting heterologous binding sites into at least two loop regions of eCGP123 or a derivative thereof, preferably in three loop positions on the same surface of the protein, thereby obtaining a fluorobody.

In another aspect, the invention provides a method of identifying a fluorobody that specifically binds to a target molecule, the method comprising, providing a library as set forth above; screening the library with the target molecule; and selecting a fluorobody that binds to the target molecule.

Another aspect of the invention provides a method of detecting the presence of an antigen in a sample, comprising incubating the sample with a fluorobody capable of specifically binding to the antigen, under conditions permitting the fluorobody to bind to the antigen if present in the sample, followed by washing unbound fluorobody from the sample, and detecting fluorescence in the sample. The detection of fluorescence in the sample thereby provides an indication of the presence of the antigen in the sample. In a related method for quantifying the level of an antigen present in a sample, the sample is incubated with a fluorobody capable of specifically binding to the antigen, under conditions permitting the fluorobody to bind to the antigen if present in the sample, followed by washing unbound fluorobody from the sample, and measuring the degree of fluorescence in the sample. The degree of fluorescence in the sample relative to a defined standard level of fluorescence generated by the binding of the fluorobody to a defined quantity of the antigen defines the quantity of antigen present in the sample.

The invention also provides a method for generating fluorobodies that are functionally equivalent to the binding characteristics of a particular monoclonal antibody. In particular, a method for generating a fluorobody recognizing a specific epitope on an antigen comprises screening a fluorobody library with the antigen, and selecting clones which bind to the antigen. The selected clones are then re-bound to the antigen. The antigen-bound clones are then contacted with an excess quantity of a monoclonal antibody which specifically recognizes the epitope, such quantity to be sufficient to elute clones bound to antigen via the same epitope. The eluted clones are then selected for generation of fluorobodies or optionally, for further selection against the antigen.

In another aspect, a method of detecting the expression of a protein of interest on a cell is provided. The cell is contacted with a fluorobody specific for the protein of interest, under conditions permitting the fluorobody to bind to the protein if expressed on the cell. Unbound fluorobody is washed from the cell, and the cell is irradiated with light corresponding to the excitation wavelength of the fluorobody. Fluorescence emitted from the cell, if detected provides and indication of expression of the protein.

Additional aspects of the invention include in vivo imaging, such as tumor imaging, using fluorobodies. For example, a method of imaging a tumor in a patient is provided, the method comprising administering a fluorobody specific for an antigen expressed in or on the tumor cell, irradiating the patient with light corresponding to the excitation wavelength of the fluorobody, and visualizing the emission of fluorescence from the tumor.

### BRIEF DESCRIPTION OF THE DRAWINGS

FIG. 1. Structure of eCGP123 in ribbon-diagrammatic. Side view, left. Top view, right. Loops in which diversity was inserted are indicated in dark black and labeled.

FIG. 2. Amino acid sequence of TGP, showing insertion sites within loops 2, 3 and 6 [SEQ ID NO: 3].

FIG. 3. The assembly strategy to create fluorobodies with three regions of diversity.

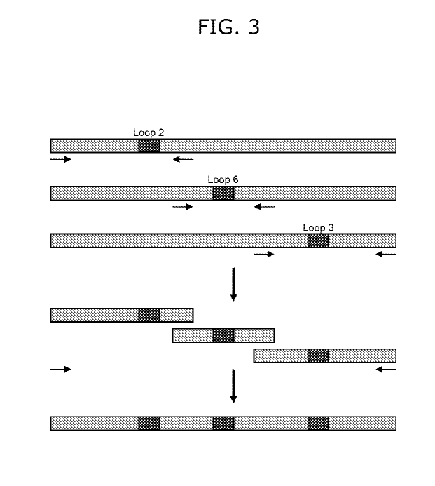

FIG. 4. Space-filling structural renderings of Thermostable Green Protein (TGP) showing diversity insertion sites at loops 2, 3 and 6, individually (upper images) and in combination (lower images, three view renderings).

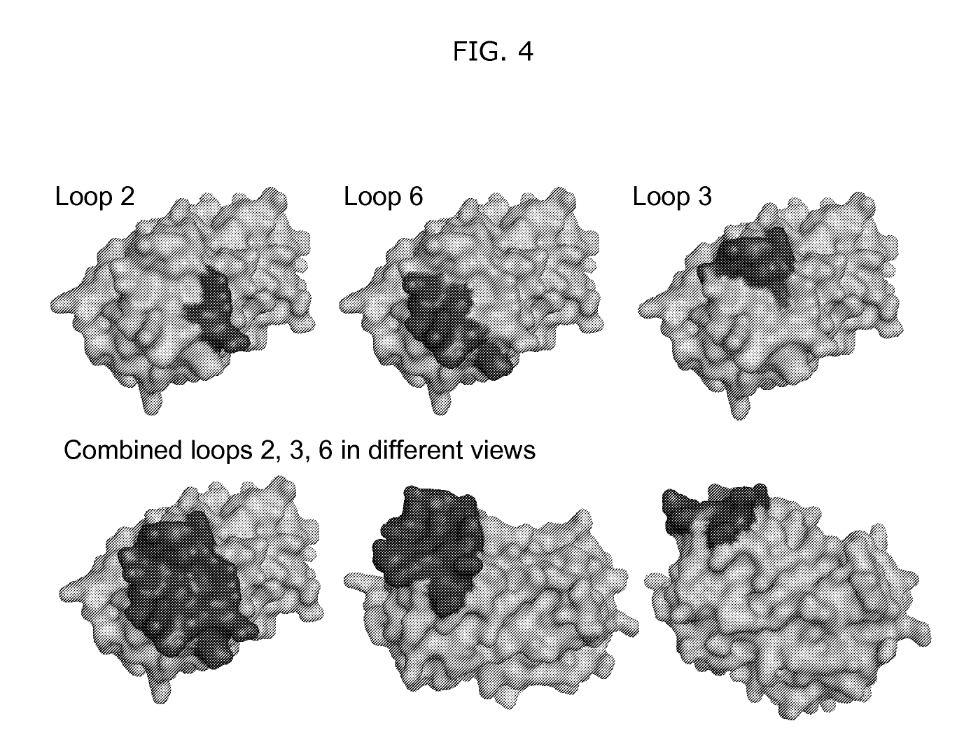

FIG. 5. TAT based display. A: the pTAT-coil plasmid construct; B: the predicted from of the displayed protein; C: fluorescent phage pellet using the TAT vector, compared to D: phage pellet with Sec based leader.

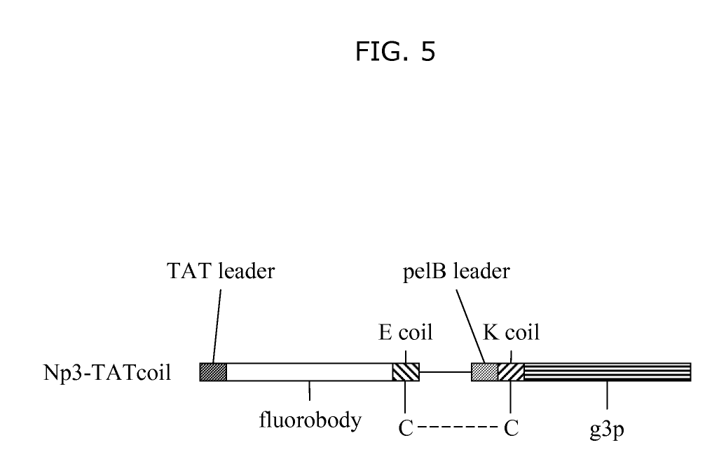

FIG. 6. ELISA evaluation of anti-lysozyme fluorobodies, using lysozyme and two irrelevant antigens (myoglobin and neutravidin). See, Example 1, infra.

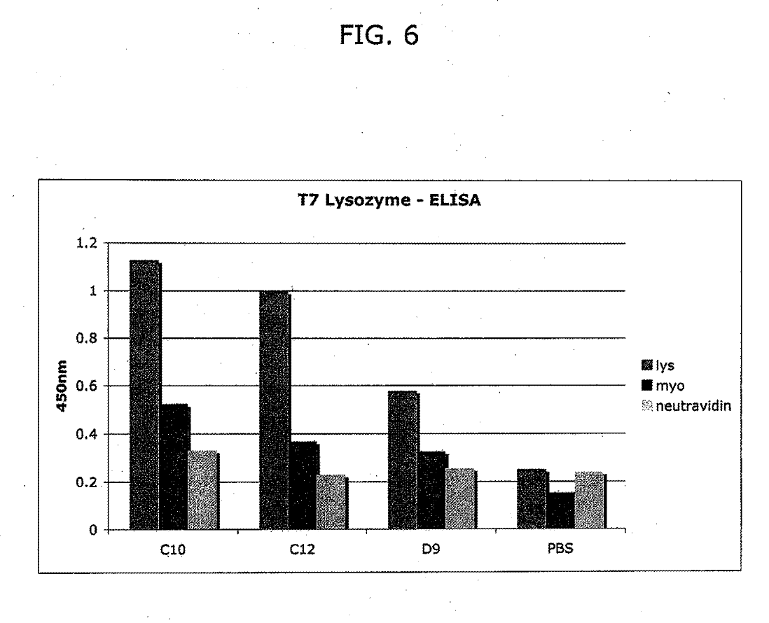

### DETAILED DESCRIPTION OF THE INVENTION

### Definitions

Unless otherwise defined, all terms of art, notations and other scientific terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art. The techniques and procedures described or referenced herein are generally well understood and commonly employed using conventional methodology by those skilled in the art, such as, for example, the widely utilized molecular cloning methodologies described in Sambrook et al., Molecular Cloning: A Laboratory Manual 3rd. edition (2001) Cold Spring Harbor Laboratory Press, Cold Spring Harbor, N.Y. and Current Protocols in Molecular Biology (Ausbel et al., eds., John Wiley & Sons, Inc. 2001. As appropriate, procedures involving the use of commercially available kits and reagents are generally carried out in accordance with manufacturer defined protocols and/or parameters unless otherwise noted.

A “fluorescent protein” as used herein is an Aequorea victoria green fluorescent protein (GFP), structural variants of GFP (i.e., circular permutants, monomeric versions), folding variants of GFP (i.e., more soluble versions, superfolder versions), spectral variants of GFP (i.e., YFP, CFP), and GFP-like fluorescent proteins (i.e., DsRed). The term “GFP-like fluorescent protein” is used to refer to members of the Anthozoa fluorescent proteins sharing the 11-beta strand “barrel” structure of GFP, as well as structural, folding and spectral variants thereof. GFP-like proteins all share common structural and functional characteristics, including without limitation, the capacity to form internal chromophores without requiring accessory co-factors, external enzymatic catalysis or substrates, other than molecular oxygen. GFP-like proteins may be defined by various structural and functional features, and have recently been pylogenetically classified into a ubiquitous Metazoan superfamily of proteins (Shagin et al., 2004, Mol. Biol. Evol. 21(5): 841-850).

A “variant” of a fluorescent protein is derived from a “parent” fluorescent protein and retains the 11 beta-strand barrel structure as well as intrinsic fluorescence, and is meant to include structures with amino acid substitutions, deletions or insertions that may impart new or modified biological properties to the protein (i.e., greater stability, improved solubility, improved folding, shifts in emission or excitation spectra, reduced or eliminated capacity to form multimers, etc) as well as structures having modified N and C termini (i.e., circular permutants).

The fluorescent protein “eCGP” refers to a class of novel, highly thermostable fluorescent proteins, described in co-owned, co-pending U.S. patent application Ser. No. 12/317,185 filed Dec. 19, 2008, the contents of which is hereby incorporated herein in its entirety. Two of the described eCGPs, “eCGP23” and “eCGP123”, are able to retain fluorescence after being exposed to very high temperatures; both are able to recover almost completely after heating at 99° C., a temperature that irreversibly destroys folding in all other fluorescent proteins tested. Similarly, both of these eCGPs are able to retain some degree of fluorescence even at the high temperature of 99° C. Additionally, both of these eCGPs retain approximately 85% of their ambient temperature fluorescence levels for at least 14 hours at 80° C. These are hitherto unreported levels of thermotolerance for fluorescent proteins. A less aggregating derivative of eCGP123, which differs in the amino acid at residue 190, is referred to herein as “TGP” (thermal green protein), the amino acid and coding sequences of which are disclosed herein as SEQ ID NOs: 3 and 4, respectively.

The term “intrinsic fluorescence” as used herein refers to the ability of a compound to emit fluorescent light upon excitation with light of the appropriate wavelength.

The “MMDB Id: 5742 structure” as used herein refers to the GFP structure disclosed by Ormo & Remington, MMDB Id: 5742, in the Molecular Modeling Database (MMDB), PDB Id: 1EMA PDB Authors: M. Ormo & S. J. Remington PDB Deposition: 1-Aug-96 PDB Class: Fluorescent Protein PDB Title: Green Fluorescent Protein From Aequorea Victoria. The Protein Data Bank (PDB) reference is Id PDB Id: 1EMA PDB Authors: M. Ormo & S. J. Remington PDB Deposition: 1 Aug. 96 PDB Class: Fluorescent Protein PDB Title: Green Fluorescent Protein From Aequorea Victoria. (see, e.g., Ormo et al. “Crystal structure of the Aequorea victoria green fluorescent protein.” Science 1996 Sep. 6; 273(5280):1392-5; Yang et al, “The molecular structure of green fluorescent protein.” Nat. Biotechnol. 1996 October; 14(10): 1246-51).

“Root mean square deviation” (“RMSD”) refers to the root mean square superposition residual in Angstroms. This number is calculated after optimal superposition of two structures, as the square root of the mean square distances between equivalent C-alpha-atoms.

A “fluorobody” as used herein refers to a polypeptide that has intrinsic fluorescence activity and specifically binds to a binding partner (e.g., antigen) via heterologous amino acid residues introduced into loop regions of a fluorescent protein, e.g., TGF, eCGP123. The fluorescent protein therefore serves as a “backbone” (or “scaffold” or “framework”) of the fluorescent binding ligand.

“FRET”, or “Fluorescence Resonance Energy Transfer”, refers to the non-radiative transfer of energy from a donor fluorophore to an acceptor fluorophore spatially located within about 80 Angstroms of each other. The relative geometric context of the two fluorophores is an important component of FRET. Circular permutation may be used to alter the geometric orientation of the fluorophores relative to each other.

A “binding site” as used herein is an amino acid sequence inserted into a loop region that specifically binds a binding partner, (e.g. antigen).

The term “heterologous” when used with reference to portions of a nucleic acid indicates that the nucleic acid comprises two or more subsequences that are not found in the same relationship to each other in nature. For instance, a nucleic acid is typically recombinantly produced, having two or more sequences from unrelated genes arranged to make a new functional nucleic acid, e.g., a nucleic acid encoding a fluorescent protein from one source and a nucleic acid encoding a peptide sequence from another source. Similarly, a heterologous protein indicates that the protein comprises two or more subsequences that are not found in the same relationship to each other in nature (e.g., a fusion protein).

The terms “identical” or percent “identity,” in the context of two or more nucleic acids or polypeptide sequences, refer to two or more sequences or subsequences that are the same or have a specified percentage of amino acid residues or nucleotides that are the same (i.e., about 70% identity, preferably 75%, 80%, 85%, 90%, or 95% identity over a specified region, when compared and aligned for maximum correspondence over a comparison window, or designated region as measured using a BLAST or BLAST 2.0 sequence comparison algorithms with default parameters described below, or by manual alignment and visual inspection. Such sequences are then said to be “substantially identical.” This definition also refers to the compliment of a test sequence. Preferably, the identity exists over a region that is at least about 22 amino acids or nucleotides in length, or more preferably over a region that is 30, 40, or 50-100 amino acids or nucleotides in length.

For sequence comparison, typically one sequence acts as a reference sequence, to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window”, as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison can be conducted, e.g., by the local homology algorithm of Smith & Waterman, 1981, Adv. Appl. Math. 2:482, by the homology alignment algorithm of Needleman & Wunsch, 1970, J. Mol. Biol. 48:443, by the search for similarity method of Pearson & Lipman, 1988, Proc. Nat'l. Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by manual alignment and visual inspection (see, e.g., Current Protocols in Molecular Biology (Ausubel et al., eds. 1995 supplement)).

A preferred example of algorithm that is suitable for determining percent sequence identity and sequence similarity are the BLAST and BLAST 2.0 algorithms, which are described in Altschul et al., 1977, Nuc. Acids Res. 25:3389-3402 and Altschul et al., 1990, J. Mol. Biol. 215:403-410, respectively. BLAST and BLAST 2.0 are used, typically with the default parameters described herein, to determine percent sequence identity for the nucleic acids and proteins of the invention. Software for performing BLAST analyses is publicly available through the National Center for Biotechnology Information. This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al., supra). These initial neighborhood word hits act as seeds for initiating searches to find longer HSPs containing them. The word hits are extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4 and a comparison of both strands. For amino acid sequences, the BLASTP program uses as defaults a wordlength of 3, and expectation (E) of 10, and the BLOSUM62 scoring matrix (see Henikoff & Henikoff, Proc. Natl. Acad. Sci. USA 89:10915 (1989)) alignments (B) of 50, expectation (E) of 10, M=5, N=−4, and a comparison of both strands.

The BLAST algorithm also performs a statistical analysis of the similarity between two sequences (see, e.g., Karlin & Altschul, 1993, Proc. Nat'l. Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.2, more preferably less than about 0.01, and most preferably less than about 0.001.

The term “as determined by maximal correspondence” in the context of referring to a reference SEQ ID NO means that a sequence is maximally aligned with the reference SEQ ID NO over the length of the reference sequence using an algorithm such as BLAST set to the default parameters. Such a determination is easily made by one of skill in the art.

The term “link” as used herein refers to a physical linkage as well as linkage that occurs by virtue of co-existence within a biological particle, e.g., phage, bacteria, yeast or other eukaryotic cell.

“Physical linkage” refers to any method known in the art for functionally connecting two molecules, including without limitation, recombinant fusion with or without intervening domains, intein-mediated fusion, non-covalent association, covalent bonding (e.g., disulfide bonding and other covalent bonding), hydrogen bonding; electrostatic bonding; and conformational bonding, e.g., antibody-antigen, and biotin-avidin associations.

“Fused” refers to linkage by covalent bonding.

As used herein, “linker” or “spacer” refers to a molecule or group of molecules that connects two molecules, such as a fluorescent binding ligand and a display protein or nucleic acid, and serves to place the two molecules in a preferred configuration.

“Antibody” refers to a polypeptide substantially encoded by an immunoglobulin gene or immunoglobulin genes, or fragments thereof which specifically bind and recognize an analyte (antigen). The recognized immunoglobulin genes include the kappa, lambda, alpha, gamma, delta, epsilon and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially an Fab with part of the hinge region (see, Paul (Ed.) Fundamental Immunology, Third Edition, Raven Press, NY (1993)). While various antibody fragments are defined in terms of the digestion of an intact antibody, one of skill will appreciate that such fragments may be synthesized de novo either chemically or by utilizing recombinant DNA methodology. Thus, the term antibody, as used herein, also includes antibody fragments either produced by the modification of whole antibodies or those synthesized de novo using recombinant DNA methodologies (e.g., single chain Fv).

The term “complementarity determining region” or “CDR” as used herein refers to the art-recognized term as exemplified by the Kabat and Chothia CDR definitions. CDRs are also generally known as hypervariable regions or hypervariable loops (Chothia and Lesk 1987 J. Mol. Biol. 196: 901; Chothia et al. (1989) Nature 342: 877; Kabat et al., Sequences of Proteins of Immunological Interest (National Institutes of Health, Bethesda, Md.) (1987); and Tramontano et al., 1990, J. Mol. Biol. 215: 175). Variable region domains typically comprise the amino-terminal approximately 105-115 amino acids of a naturally-occurring immunoglobulin chain (e.g., amino acids 1-110), although variable domains somewhat shorter or longer are also suitable for forming single-chain antibodies.

As used herein, “random peptide sequence” refers to an amino acid sequence composed of two or more amino acid monomers and constructed by a stochastic or random process. A random peptide can include framework or scaffolding protein sequences, e.g., GFP protein sequences, that may comprise invariant sequences.

The terms “polypeptide,” “peptide” and “protein” are used interchangeably herein to refer to a polymer of amino acid residues. The terms apply to amino acid polymers in which one or more amino acid residue is an artificial chemical mimetic of a corresponding naturally occurring amino acid, as well as to naturally occurring amino acid polymers and non-naturally occurring amino acid polymer.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refers to compounds that have the same basic chemical structure as a naturally occurring amino acid, i.e., an α carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The term “binding polypeptide” or “binding ligand” as used herein refers to a polypeptide that specifically binds to a target molecule (e.g. an antigen). Although a binding ligand may comprises a region from an immunoglobulin fragment, such as a CDR, binding polypeptides are typically distinguished from antibodies in that binding polypeptides do not usually have the same structural fold as immunoglobulins, or immunoglobulin fragments, although some, such as those based on CTLA4, are similar.

A “target molecule” in the context of this invention may be any molecule that will selectively bind to a fluorescent binding ligand of the invention. Typically, the target molecule is a protein, such as an antigen, or a receptor and the like, but may also be a non-protein molecule, e.g., a carbohydrate or lipid, haptens, organic molecules, small molecule pharmaceuticals, post-translational modifications occurring on polypeptides.

The term “nucleic acid” refers to deoxyribonucleotides or ribonucleotides and polymers thereof in either single- or double-stranded form. Unless specifically limited, the term encompasses nucleic acids containing known analogues of natural nucleotides which have similar binding properties as the reference nucleic acid and are metabolized in a manner similar to naturally occurring nucleotides. Unless otherwise indicated, a particular nucleic acid sequence also implicitly encompasses conservatively modified variants thereof (e.g. degenerate codon substitutions) and complementary sequences and as well as the sequence explicitly indicated. Specifically, degenerate codon substitutions may be achieved by generating sequences in which the third position of one or more selected (or all) codons is substituted with mixed-base and/or deoxyinosine residues (Batzer et al., 1991, Nucleic Acid Res. 19: 5081; Ohtsuka et al., 1985 J. Biol. Chem. 260: 2605-2608; and Cassol et al., 1992; Rossolini et al., 1994, Mol. Cell. Probes 8: 91-98). The term nucleic acid is used interchangeably with gene, cDNA, and mRNA encoded by a gene.

“Conservatively modified variants” applies to both amino acid and nucleic acid sequences. With respect to particular nucleic acid sequences, conservatively modified variants refers to those nucleic acids which encode identical or essentially identical amino acid sequences, or where the nucleic acid does not encode an amino acid sequence, to essentially identical sequences. Because of the degeneracy of the genetic code, a large number of functionally identical nucleic acids encode any given protein. For instance, the codons GCA, GCC, GCG and GCU all encode the amino acid alanine. Thus, at every position where an alanine is specified by a codon, the codon can be altered to any of the corresponding codons described without altering the encoded polypeptide. Such nucleic acid variations are “silent variations,” which are one species of conservatively modified variations. Every nucleic acid sequence herein which encodes a polypeptide also describes every possible silent variation of the nucleic acid. One of skill will recognize that each codon in a nucleic acid (except AUG, which is ordinarily the only codon for methionine, and TGG, which is ordinarily the only codon for tryptophan) can be modified to yield a functionally identical molecule. Accordingly, each silent variation of a nucleic acid which encodes a polypeptide is implicit in each described sequence.

As to amino acid sequences, one of skill will recognize that individual substitutions, deletions or additions to a nucleic acid, peptide, polypeptide, or protein sequence which alters, adds or deletes a single amino acid or a small percentage of amino acids in the encoded sequence is a “conservatively modified variant” where the alteration results in the substitution of an amino acid with a chemically similar amino acid. Conservative substitution tables providing functionally similar amino acids are well known in the art. Such conservatively modified variants are in addition to and do not exclude polymorphic variants, interspecies homologs, and alleles of the invention.

The following eight groups each contain amino acids that are conservative substitutions for one another: 1) Alanine (A), Glycine (G); 2) Aspartic acid (D), Glutamic acid (E); 3) Asparagine (N), Glutamine (Q); 4) Arginine (R), Lysine (K); 5) Isoleucine (I), Leucine (L), Methionine (M), Valine (V); 6) Phenylalanine (F), Tyrosine (Y), Tryptophan (W); 7) Serine (S), Threonine (T); and 8) Cysteine (C), Methionine (M) (see, e.g., Creighton, Proteins (1984)).

Macromolecular structures such as polypeptide structures can be described in terms of various levels of organization. For a general discussion of this organization, see, e.g., Alberts et al., Molecular Biology of the Cell (3^{rd} ed., 1994) and Cantor and Schimmel, Biophysical Chemistry Part I: The Conformation of Biological Macromolecules (1980). “Primary structure” refers to the amino acid sequence of a particular peptide. “Secondary structure” refers to locally ordered, three dimensional structures within a polypeptide. These structures are commonly known as domains. Domains are portions of a polypeptide that form a compact unit of the polypeptide and are typically 25 to approximately 500 amino acids long. Typical domains are made up of sections of lesser organization such as stretches of -sheet and -helices. “Tertiary structure” refers to the complete three dimensional structure of a polypeptide monomer. “Quaternary structure” refers to the three dimensional structure formed by the noncovalent association of independent tertiary units. Anisotropic terms are also known as energy terms.

The terms “isolated” or “biologically pure” refer to material which is substantially or essentially free from components which normally accompany it as found in its native state. However, the term “isolated” is not intended refer to the components present in an electrophoretic gel or other separation medium. An isolated component is free from such separation media and in a form ready for use in another application or already in use in the new application/milieu.

As used herein “random peptide library” refers to a set of polynucleotide sequences that encodes a set of random peptides, and to the set of random peptides encoded by those polynucleotide sequences, as well as the fusion proteins containing those random peptides.

As used herein, “CDR library” refers to a set of polynucleotide sequences that encode CDR regions and to the set of CDR polypeptide sequences encoded by those polynucleotide sequences, as well as the fusion proteins containing the CDR sequences.

The phrase “specifically (or selectively) binds” to a binding partner, e.g., an antigen, or “specifically (or selectively) reactive with,” when referring to a protein or peptide, refers to a binding reaction that is determinative of the presence of the protein in a heterogeneous population of proteins and other biologics. Thus, under designated assay conditions, the specified antigen binds to a particular protein above background, e.g., at least two times the background, and does not substantially bind in a significant amount to other proteins present in the sample. Typically a specific or selective reaction will be at least twice background signal or noise and more typically more than 10 to 100 times background. Specific binding to an antibody under these conditions may require an antibody that is selected for its specificity for a particular protein. For example, polyclonal antibodies raised to a particular protein or antigen can be selected to obtain only those polyclonal antibodies that are specifically immunoreactive with the antigen, and not with other proteins, except for polymorphic variants, orthologs, and alleles of the protein. This selection may be achieved by subtracting out antibodies that cross-react with the antigen. A variety of immunoassay formats may be used to select antibodies specifically immunoreactive with a particular protein. For example, solid-phase ELISA immunoassays are routinely used to select antibodies specifically immunoreactive with a protein (see, e.g., Harlow & Lane, Antibodies, A Laboratory Manual (1988), for a description of immunoassay formats and conditions that can be used to determine specific immunoreactivity).

The term “population” as used herein means a collection of components such as polynucleotides, portions or polynucleotides or proteins. A “mixed population: means a collection of components which belong to the same family of nucleic acids or proteins (i.e., are related) but which differ in their sequence (i.e., are not identical) and hence in their biological activity.

A “display vector” refers to a vector used to create a cell or virus that displays, i.e., expresses a display protein comprising a heterologous polypeptide, on its surface or in a cell compartment such that the polypeptide is accessible to test binding to target molecules of interest, such as antigens.

A “display library” refers to a population of display vehicles, often, but not always, cells or viruses. The “display vehicle” provides both the nucleic acid encoding a peptide as well as the peptide, such that the peptide is available for binding to a target molecule and further, provides a link between the peptide and the nucleic acid sequence that encodes the peptide. Various “display libraries” are known to those of skill in the art and include libraries such as phage, phagemids, yeast and other eukaryotic cells, bacterial display libraries, plasmid display libraries as well as in vitro libraries that do not require cells, for example ribosome display libraries or mRNA display libraries, where a physical linkage occurs between the mRNA or cDNA nucleic acid, and the protein encoded by the mRNA or cDNA.

A “phage expression vector” or “phagemid” refers to any phage-based recombinant expression system for the purpose of expressing a nucleic acid sequence in vitro or in vivo, constitutively or inducibly, in any cell, including prokaryotic, yeast, fungal, plant, insect or mammalian cell. A phage expression vector typically can both reproduce in a bacterial cell and, under proper conditions, produce phage particles. The term includes linear or circular expression systems and encompasses both phage-based expression vectors that remain episomal or integrate into the host cell genome.

A “phage display library” refers to a “library” of bacteriophages on whose surface is expressed exogenous peptides or proteins. The foreign peptides or polypeptides are displayed on the phage capsid outer surface. The foreign peptide can be displayed as recombinant fusion proteins incorporated as part of a phage coat protein, as recombinant fusion proteins that are not normally phage coat proteins, but which are able to become incorporated into the capsid outer surface, or as proteins or peptides that become linked, covalently or not, to such proteins. This is accomplished by inserting an exogenous nucleic acid sequence into a nucleic acid that can be packaged into phage particles. Such exogenous nucleic acid sequences may be inserted, for example, into the coding sequence of a phage coat protein gene. If the foreign sequence is “in phase” the protein it encodes will be expressed as part of the coat protein. Thus, libraries of nucleic acid sequences, such as a genomic library from a specific cell or chromosome, can be so inserted into phages to create “phage libraries.” As peptides and proteins representative of those encoded for by the nucleic acid library are displayed by the phage, a “peptide-display library” is generated. While a variety of bacteriophages are used in such library constructions, typically, filamentous phage are used (Dunn, 1996 Curr. Opin. Biotechnol. 7:547-553). See, e.g., description of phage display libraries, below.

The term “amplification” means that the number of copies of a polynucleotide is increased.

### Structural, Physical, and Biological Characteristics:

The fluorobodies of the invention offer a number of distinct advantages over the combination of antibodies and secondary detection agents presently in widespread use in biological medicine and research, including particularly, intrinsic fluorescence or color, very high stability relative to antibodies and antibody derivative reagents, as well as the binding specificity and sensitivity typical of monoclonal antibodies.

Monoclonal antibodies are stable proteins, of high affinity and specificity, which can be used in many research procedures. However, antibody generation is time consuming, labor intensive and requires mouse immunization. Ten years ago it seemed likely that monoclonal antibodies would be replaced by single chain Fvs (scFvs) or Fabs selected from large naïve phage display libraries, because they appeared to offer the advantages of diversity, high affinity and specificity in a potentially high throughput format, which also avoided the use of animals and the problems of poor immunogenicity. While scFvs have been very successful in some cases, it has been found that their use beyond simple ELISAs is often limited by low production levels, relatively poor stability, and the need for additional labeling steps.

In view of the ineffective and inconsistent use scFvs, applicants examined alternative scaffolds and developed the binding ligands of the present invention to address these limitations of known binding ligands. The binding ligands of the invention combine the advantages of monoclonal antibodies (specific, sensitive, high affinity binding) with those of the fluorescent proteins, such as Green Fluorescent Protein from the bioluminescent jellyfish Aequorea victoria (intrinsic fluorescence, high expression, stability and solubility) and related fluorescent proteins.

In preferred embodiments, eCGP fluorescent proteins are used as the scaffold for generating the fluorobodies of the invention. More preferably, the most thermostable of the eCGP fluorescent proteins, eCGP123, or a derivative thereof, is used as the scaffold in the generation of the fluorobodies of the invention. One particular derivative of eCGP123, termed “thermal green protein” (“TGP”, see SEQ ID NO: 3) differs at one amino acid residue from eCGP123 (190, lysine, in TGP). The change to the lysine residue from the glutamate residue in eCGP123, mirrors the lysine at the same residue in the CGP molecule from which eCGP123 was derived. The reversion to lysine results in reduced aggregation compared to eCGP123, but has no adverse impact on stability. TGP was used in the generation of the fluorobodies as further described in the Examples, infra.

In further preferred embodiments, variants of eCGP123 which do not exhibit aggregation upon expression are used to prepare the fluorobodies of the invention. In one embodiment, the nsTGP(−1) protein [SEQ ID NO: 5] is utilized. This protein replaces the C-terminal seven amino acid residues in the eCGP123 structure with the sequence gly-gly-gly-ser-gly-gly-gly. In another embodiment, the nsTGP(−10) protein is utilized. This protein includes a higher proportion of

In the first, the last seven amino acids of the eCGP structure were replaced with the sequence gly-gly-gly-ser-gly-gly-gly [SEQ ID NO: 5]. In the second and third [SEQ ID NOS: 7 and 9], the negative charge of eCGP123 was increased by including more negatively amino acids. The three improved eCGP proteins, termed nsTGP(−1), nsTGP(−10) and nsTGP(−18) were expressed and evaluated for agregation tendencies, in comparison to eCGP. All three variants showed no aggregation after dialysis into PBS and exhibited very similar thermostability.

The fluorobodies of the invention are highly stable proteins, and may be considered to be robust, well-expressed antibodies with intrinsic fluorescence, amenable to high throughput selection.

As described in further detail below, the high degree of stability of fluorobodies is due in part from the inherent stability of the scaffold used to generate these unique binding ligands, in particular, the fluorescent proteins used as the scaffold retain critical structural elements of the fluorescent protein family, all of which are generally characterized by an 11-stranded beta-barrel structure, surrounding a coaxial central helix containing an autocatalytic chromophore-forming amino acid sequence. The fluorescent scaffold used in the practice of the invention may be heated to 90° C. and regain complete functionality in a matter of seconds. After incubation at 80° C. for 14 hours, it loses only 15% fluorescence.

Functionally, fluorobodies incorporate the advantages of antibodies as binding ligands. However, unlike antibodies or indeed any known binding ligand, the fluorobodies of the invention also have the property of intrinsic fluorescence enabling them to be directly visualized and detected by the emission of characteristic light or color. In the case of fluorobodies, for example, this property permits visual tracking through all phases of fluorobody generation, screening and selection, without the need for secondary detection reagents and methods.

The solubility characteristics of fluorobodies also provide distinct advantages in production in comparison to antibodies. In particular, fluorobodies may be expressed in the cytoplasm as well as the periplasm of host cells, and can correctly fold extracellularly. In contrast, antibodies and antibody derivatives, such as single chain antibodies, may only be expressed in the periplasm or within the secretory compartment of eukaryotic cells, unless they are specifically evolved to possess the greater stability required for functional cytoplasmic expression In addition, fluorobodies are expressed at very high levels compared to all antibody and antibody-derived reagents. Together, these characteristics provide lower costs of production and use in comparison to antibody reagents.

Fluorobodies also have a number of other characteristics which are likely to be very useful in biomedical research and molecular diagnostics and medicine. For example, fluorobodies are smaller molecules relative to antibodies (about one-sixth the size of antibodies). This characteristic, and the particular can-like structure, may permit fluorobodies to gain intracellular access without the need for additional targeting signals or cellular permeabilization. Additionally, fluorobodies with variable emission spectra may be designed by reference to the known spectral properties of either natural or mutated fluorescent proteins. Such fluorobodies may be used productively in FRET based methods as has already been shown for intracellular proteins tagged with fluorescent proteins of different colors (e.g. blue and green, or cyan and yellow fluorescent proteins). When such proteins interact with one another, the attached fluorescent proteins are able to exhibit FRET. Similarly, fluorobodies of different (and FRETable) colors recognizing different epitopes of a single target are likely to undergo FRET when binding simultaneously.

### Generation of Fluorobodies:

### A. Generation and Selection of a Scaffold Fluorescent Protein

An examination of the literature reveals three steps generally used to transform a protein without any binding activity into one with the potential for binding activity: 1) Guided by the structure, identify the sites at which diversity is to be introduced—preferably exposed loops or surfaces on one face of the protein. In general, insertions at three sites have been used to derive high affinity binders; 2) Engineer a more stable protein—consensus sequences have often been used for this step²⁵; and 3) Choose the form of diversity. This has been either replacement diversity^{9,26,27,28,29,30,31,32,33}—where amino acids present in the scaffold of interest are randomized—or insertional diversity, where stretches of random amino acids are introduced to a specific Site^{34,35,36,37,38}. Specific affinity reagents recognizing targets of interest are then selected from such libraries within the context of a display platform, with phage, yeast and ribosome display being the most commonly used. For any specific affinity reagent scaffold, a suitable display platform must provide: 1) expression and correct folding of the scaffold; 2) coupling of phenotype (protein) to genotype (gene) so that selection of a scaffold on the basis of binding activity will automatically lead to selection of the gene that encodes it; and 3) the ability to carry out amplification after selection. Because of the different folding requirements of different proteins, not all display platforms are suitable for all proteins, and it is important to test any potential scaffold within the context of appropriate display platforms.

In order to create fluorescent recognition elements, it was hypothesized that it is essential to start with a fluorescent protein that was even more stable than superfolder GFP (sfGFP) ¹⁸. Furthermore, rather than the general increase in stability manifested by sfGFP, such a fluorescent protein should be able to specifically resist the detrimental effects on folding and stability mediated by inserts such as binding loops. Pursuant to this, applicants created a protein stabilization strategy which involved internally destabilizing a fluorescent protein by the insertion of an antibody binding loop (see U.S. patent application Ser. No. 12/317,185, filed Dec. 19, 2008, for details). This internal destabilization resulted in loss of fluorescence. The fluorescent protein was then evolved to overcome the destabilization and recover fluorescence. This was repeated three times until a protein that could resist the effects of three destabilizing inserts was obtained. For each insert three to four rounds of evolution were required. Applicants believe that when evolved to overcome such destabilizing insults, new internal non-covalent bonds and folding pathways are introduced that greatly enhance protein stability.

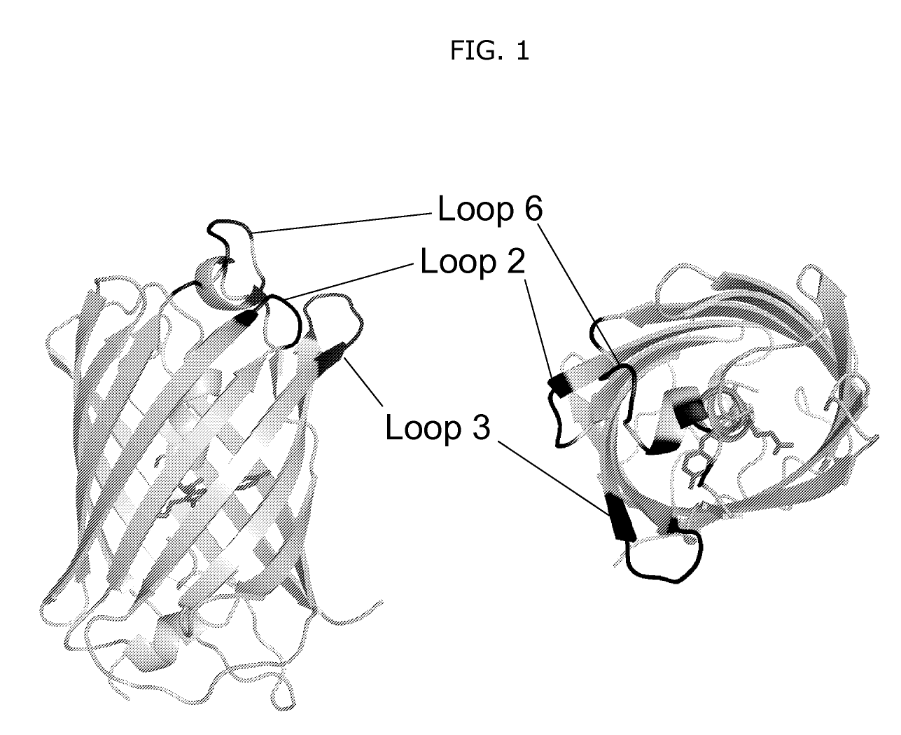

The foregoing method was applied to a consensus green protein (CGP), recently described²³, to create the astonishingly stable protein, eCGP123. The eCGP protein is so stable that when the inserts were removed, the resulting fluorescent protein could be boiled briefly and retain fluorescence, and could be incubated at 80° C. for fourteen hours losing less than 15% of its fluorescence. In collaboration with Dr. Mark Prescott, Monash University, Australia, the structure of eCGP123 was determined (FIG. 1), allowing the determination of specific sites for mutation, as well as amino acids close to the fluorophore. This structure also permitted the generation of the conserved TGP variant of eCGP123, which has a reduced tendency to aggregate in relation to eCGP.

### B. Creating a Suitable Display Platform

In addition to a stable scaffold, selection of specific binding clones requires a suitable display platform. Fluorescent proteins are expressed, and fold, in the cytoplasm. As a consequence, applicants have found that display platforms, based on standard filamentous phage display, which involve folding in the periplasmic compartment, are not suitable.

Most proteins crossing the inner bacterial membrane do so using the type II secretory system. This comprises three different pathways: Sec, SRP (signal recognition particle) and TAT (twin arginine transporter). The Sec pathway translocates proteins post-translationall^{40,41,42}, the SRP pathway translocates co-translationally^{43,44}, and both pathways converge at the Sec translocon, which transports proteins in an unfolded state across the inner membrane. This is in contrast to the TAT pathway which only translocates proteins that have already folded in the cytoplasm across the inner membrane^{45,46,47,48}. Traditional phage display vectors, such as those used to select antibody fragments, use the Sec leader to translocate the unfolded antibody fragments into the periplasm, where they subsequently fold. The TAT pathway has been widely used to transport folded and fluorescent GFP from the cytoplasm to the periplasm^{49,50,51,52,53}, as well as in a genetic selection system for correctly folded proteins⁵⁴. Recently, in attempts to display otherwise undisplayable proteins, SRP⁵⁵ and TAT⁵⁶ leaders were used in phage display constructs. The SRP leader was used to display ankyrin based binding proteins, while the TAT leader was used to display a circular GFP permutant. In both cases, the previous use of standard phage display vectors with the Sec leader was unsuccessful. Specific ankyrin based binders could be selected from phage libraries using SRP, but not from similar Sec based libraries, while in the case of the TAT based display vector, a peptide linker fused to a circular permutant of GFP could be specifically recognized on phage. However, the display of fluorescent GFP was not demonstrated, suggesting that correctly folded and functional GFP was not displayed in this latter study.

Applicants reasoned that if a different format was used, the TAT leader could be used to display functional fluorescent proteins. Consequently, a number of different constructs were designed and evaluated, the most successful of which were pHUGH3, pHUGH5 and pHUGH7 (Velappan et al., 2009, Nucleic Acids Research 1-16). In pHUGH3, the fluorescent protein is translocated into the periplasmic space using the TAT leader. An E-coil, that has an extremely high affinity (64 pM) for a partner K-coil, is fused to the C terminus of the fluorescent protein. The display protein (g3p) is translocated across the bacterial inner membrane using a Sec leader and has a K coil fused to its N terminus. This allows the fluorescent protein to bind to the g3p display protein once both have been translocated into the periplasm. The relative success of this system relies on the fact that each protein is expressed and folded in the most suitable cellular compartment, and then translocated into the periplasm using the most suitable leader sequence. Once both proteins are in the periplasm, where filamentous phage assembly occurs, interaction between the E and K coils can occur, linking the fluorescent protein to the phage (via g3p) containing the gene that encodes it, so coupling phenotype and genotype.

The pHUGH3 vector is able to display different fluorescent proteins, including a modified GFP containing a destabilizing insert, far more effectively than standard phage display vectors⁵⁷. However, although the E/K coil interaction is of high affinity, it remains a non-covalent interaction that may theoretically be susceptible to chain switching, in which a fluorobody attached to one phage is exchanged with a different fluorobody on another phage (however, this has not been directly observed). This potential breakdown in the phenotype/genotype coupling could lead to significantly less efficient selection, as a result of the amplification of phage containing genes that do not encode fluorobodies binding to the target of interest. This potential impact of such a problem was overcome by inserting a cysteine into the center of the E and K coils in two further vector derivatives, pHUGH5 and pHUGH7. These vectors differ only in the cloning site at the 5′ end. pHUGH5 uses BssHII, while pHUGH7 uses NcoI. As a result after translocation across the inner membrane, the E and K coils interact, so aligning the two cysteines and forming a covalent disulfide bond. In this way otherwise undisplayable cytoplasmically folding proteins can be displayed on the surface of filamentous phage in a covalent fashion. In addition to using this vector system, T7 vector systems may be used, a lytic phage that has been engineered to be suitable for the display of cytoplasmic proteins.

### C. Generating Suitable Diversity

Affinity reagents have been created using replacement diversity^{9,26,27,28,29,30,31,32,33}—where amino acids present in the scaffold of interest are randomized—or insertional diversity, where stretches of random amino acids are introduced to a specific site^{34,35,36,37,38}. The introduced diversity can be completely random, encoded by, for example, oligonucleotides with the sequence (NNK)n, or composed of amino acid subsets. Recent work has shown that antibodies with affinities less than 5 nM can be selected from antibody libraries in which diversity is restricted to only two—tyrosine and serine (YS)⁵⁸, three—tyrosine, glycine and serine (YGS)⁵⁹ or four—tyrosine, alanine, aspartate and serine (YADS)⁶⁰ amino acids. The best affinity antibodies were isolated routinely from the YGS libraries. Furthermore, and particularly appropriate for the fluorobodies of the invention, antibodies isolated from such three amino acid restricted libraries showed significantly less cross-reactivity to irrelevant targets than other antibodies containing more amino acids. This approach is not restricted to antibodies, since similarly restricted libraries have also been created within the context of a fibronectin scaffold⁶¹. Diversity can also be harvested from natural sources, such as antibody binding loops and inserted into fluorescent proteins, as described in ^{17,62}. In generating the fluorobodies described in Example 1, infra, a combination of insertional and replacement diversity was inserted at three sites.

Fluorobodies are generated by the insertion of heterologous peptide sequences at the loop regions of the scaffold protein (i.e., TGP). A loop sequence is defined as the solvent-exposed peptide sequence connecting two beta strands, a beta strand and an alpha helix or two helices contiguous in primary sequence. In the current invention, loop sequences are typically determined with reference to the Ormo & Remington GFP structure (MMDB ID:5742); or with reference to SEQ ID NO: 1 (eCGP123) or SEQ ID NO: 3 (TGP). In determining the loop sequence with respect to MMDB ID:5742, the loop sequences are readily identified by those of skill in the art by visual comparison of the superimposed structures.

In some embodiments, the fluorobodies of the invention utilize diversity inserted into at least two, preferably three loops on the same surface of the scaffold, in order to provide binding specificity to the fluorobody, and diversity to fluorobody libraries. In other embodiments, fluorobodies utilize the highly evolved human complementarity determining regions of human antibodies to provide binding specificity. For example, such inserted sequences may be located in two or three of the loops 2, 3 and 6 as shown in FIG. 1. Heterologous peptide sequences may, however, be inserted in any of the loops. Preferably, the sequences are inserted in at least two loops that are on the same face of the scaffold protein.

The peptide sequences that are inserted into the loop regions, the “binding sites” can be any number of amino acids in length. Typically, the sequences are at least 2 amino acids, and may be as large as fifty or more amino acids (antibody CDRs usually range from about 2 to about 32 amino acids). Longer sequences can also be accommodated, provided their N and C termini can be brought close together.

The sequences inserted into the loop can be from any source. The sequences inserted into the loop regions maybe random peptide sequences, comprising all 20 amino acids of the genetic code, restricted sets of such random amino acids (see, supra), or CDR sequences from many different antibodies. A preferred embodiment uses a restricted set of amino acids, comprising tyrosine, glycine and serine.

In preferred embodiments, a library of fluorescent binding ligands is created in which a populations of random peptide, restricted peptide sequences or a population of CDR sequences is generated and inserted into the loop regions. The sequences at each loop region of a particular fluorescent binding ligand is therefore typically different. Such libraries can then be screened with an antigen to identifying fluorescent binding ligands that specifically bind the antigen. Typically, libraries are generated using PCR in conjunction with other standard methodology in the art.

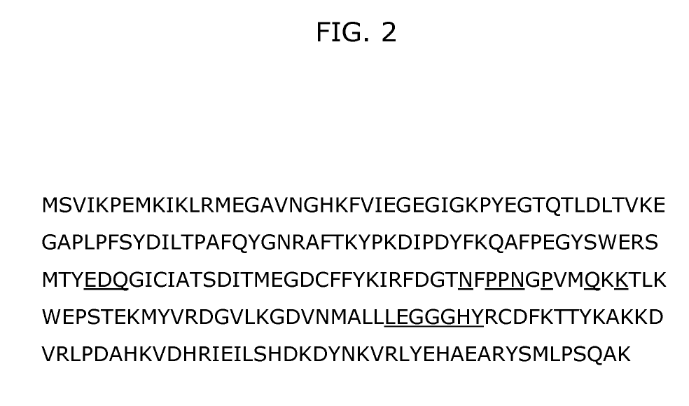

In a preferred embodiment, with reference to FIG. 2, the following TGP loops are selected for the insertion of diversity via a restricted set of random peptides comprising only three amino acids, tyrosine, glycine and serine: Loop 2: 96/97, loop 3: 164/165 and loop 6: 124-135 (referencing FIG. 2, in the following order: loops 2, 6, 3 as underlined). See Example 1 for details concerning the generation of fluorobody library using this approach, as well as the selection and characterization of fluorobodies against a test target protein, infra.

### General Nucleic Acid Methodology:

The libraries and fluorobodies of the invention are generated using basic nucleic acid methodology that is routine in the field of recombinant genetics. Basic texts disclosing the general methods of obtaining and manipulating nucleic acids in this invention include Sambrook and Russell, Molecular Cloning, a Laboratory Manual (3rd ed. 2001) and Current Protocols in Molecular Biology (Ausubel et al., eds., John Wiley & Sons, Inc. 1994-1997, 2001 version)).

Typically, the nucleic acid sequences encoding fluorobodies are generated using amplification techniques. Examples of techniques sufficient to direct persons of skill through in vitro amplification methods are found in Berger, Sambrook, and Ausubel, as well as Dieffenfach & Dveksler, PCR Primers: A Laboratory Manual (1995): Mullis et al., (1987); U.S. Pat. No. 4,683,202; PCR Protocols A Guide to Methods and Applications (Innis et al., eds) Academic Press Inc. San Diego, Calif. (1990); (Innis); Arnheim & Levinson (Oct. 1, 1990) C&EN 36-47; The Journal Of NIH Research, 1991, 3: 81-94; Kwoh et al., 1989, Proc. Natl. Acad. Sci. USA, 86: 1173; Guatelli et al., 1990, Proc. Natl. Acad. Sci. USA, 87, 1874; Lomeli et al., 1989, J. Clin. Chem., 35: 1826; Landegren et al., 1988, Science, 241: 1077-1080; Van Brunt, 1990, Biotechnology, 8: 291-294; Wu and Wallace, 1989, Gene, 4: 560; and Barringer et al., 1990, Gene 89: 117.

Amplification techniques can typically be used to obtain a population of sequences, e.g., random peptide sequences or CDRs, to insert into the loop regions. In generating a population of CDRs, it is often desirable to obtain CDRs that do not include the primer sequences from the amplification primers. This can be achieved by using primers that include restriction enzyme sites, such as Bpml, that cleave at a distance from the recognition sequence. The amplified population can then be introduced into the fluorescent protein backbone at the desired loop sites, for example, using appropriate adaptors and additional amplification reactions. See for example, Kiss et al., 2006, Nucl. Acids Res. 34: e132.

Random peptides can also be inserted into the loop regions of the fluorescent protein. The random peptides are inserted using methods well known in the art. For example, single-stranded, UTP-substituted DNA from a phagemid can be performed in which oligonucleotides that hybridize to the sequence encoding a loop region of the fluorescent protein are used. The oligonucleotides are flanked by a region of homology, for example, 21 base pairs, on either side of the insertion site and contain random based to encode the random amino acids.

### Display Libraries:

Fluorobody libraries may be constructed using a number of different display systems. In cell or virus-based systems, the ligand can be displayed, for example, on the surface of a particle, e.g., a virus or cell and screened for the ability to interact with other molecules, e.g., a library of target molecules. In vitro display systems can also be used, in which the binding ligand is linked to an agent that provides a mechanism for coupling the binding ligand to the nucleic acid sequence that encodes it. These technologies include ribosome display and mRNA display.

As noted above, in some instances, for example, ribosomal display, a fluorobody is linked to the nucleic acid sequence through a physical interaction, for example, with a ribosome. In other embodiments, e.g., mRNA display, the fluorobody may be joined to another molecule via a linking group. The linking group can be a chemical crosslinking agent, including, for example, succinimidyl-(N-maleimidomethyl)-cyclohexane-1-carboxylate (SMCC). The linking group can also be an additional amino acid sequence(s), including, for example, a polyalanine, polyglycine or similar linking group. Other near neutral amino acids, such as Ser can also be used in the linker sequence. Amino acid sequences which may be usefully employed as linkers include those disclosed in Maratea et al., 1985, Gene 40:39-46; Murphy et al., 1986, Proc. Natl. Acad. Sci. USA 83:8258-8262; U.S. Pat. Nos. 4,935,233 and 4,751,180.

Other chemical linkers include carbohydrate linkers, lipid linkers, fatty acid linkers, polyether linkers, e.g., PEG, etc. For example, poly(ethylene glycol) linkers are available from Shearwater Polymers, Inc. Huntsville, Ala. These linkers optionally have amide linkages, sulfhydryl linkages, or heterofunctional linkages.

### Phage Display Libraries:

Construction of phage display libraries exploits the bacteriophage's ability to display peptides and proteins on their surfaces, i.e., on their capsids. Often, filamentous phage such as M13, fd, or f1 are used. Filamentous phage contain single-stranded DNA surrounded by multiple copies of genes encoding major and minor coat proteins, e.g., pill. Coat proteins are displayed on the capsid's outer surface. DNA sequences inserted in-frame with capsid protein genes are co-transcribed to generate fusion proteins or protein fragments displayed on the phage surface. Phage libraries thus can display peptides representative of the diversity of the inserted sequences. Significantly, these peptides can be displayed in “natural” folded conformations. The fluorobodies expressed on phage display libraries can then bind target molecules, i.e., they can specifically interact with binding partner molecules such as antigens, e.g., (Petersen, 1995, Mol. Gen. Genet., 249:425-31), cell surface receptors (Kay, 1993, Gene 128:59-65), and extracellular and intracellular proteins (Gram, 1993, J. Immunol. Methods, 161:169-76).

The concept of using filamentous phages, such as M13 or fd, for displaying peptides on phage capsid surfaces was first introduced by Smith, 1985, Science 228:1315-1317. Peptides have been displayed on phage surfaces to identify many potential ligands (see, e.g., Cwirla, 1990, Proc. Natl. Acad. Sci. USA, 87:6378-6382). There are numerous systems and methods for generating phage display libraries described in the scientific and patent literature, see, e.g., Sambrook and Russell, Molecule Cloning: A Laboratory Manual, 3rd edition, Cold Spring Harbor Laboratory Press, Chapter 18, 2001; Phage, Display of Peptides and Proteins: A Laboratory Manual, Academic Press, San Diego, 1996; Crameri, 1994, Eur. J. Biochem. 226:53-58; de Kruif, 1995, Proc. Natl. Acad. Sci. USA, 92:3938-42; McGregor, 1996, Mol. Biotechnol., 6:155-162; Jacobsson, 1996, Biotechniques, 20:1070-1076; Jespers, 1996, Gene, 173:179-181; Jacobsson, 1997, Microbiol Res., 152:121-128; Fack, 1997, J. Immunol. Methods, 206:43-52; Rossenu, 1997, J. Protein Chem., 16:499-503; Katz, 1997, Annu. Rev. Biophys. Biomol. Struct., 26:27-45; Rader, 1997, Curr. Opin. Biotechnol., 8:503-508; Griffiths, 1998, Curr. Opin. Biotechnol., 9:102-108.

Typically, exogenous nucleic acids encoding the protein sequences to be displayed are inserted into a coat protein gene, e.g. gene III or gene VIII of the phage. The resultant fusion proteins are displayed on the surface of the capsid. Protein VIII is present in approximately 2700 copies per phage, compared to 3 to 5 copies for protein III (Jacobsson (1996), supra). Multivalent expression vectors, such as phagemids, can be used for manipulation of the nucleic acid sequences encoding the fluorescent binding library and production of phage particles in bacteria (see, e.g., Felici, 1991, J. Mol. Biol., 222:301-310).

Phagemid vectors are often employed for constructing the phage library. These vectors include the origin of DNA replication from the genome of a single-stranded filamentous bacteriophage, e.g., M13 or f1 and require the supply of the other phage proteins to create a phage. This is usually supplied by a helper phage which is less efficient at being packaged into phage particles. A phagemid can be used in the same way as an orthodox plasmid vector, but can also be used to produce filamentous bacteriophage particle that contain single-stranded copies of cloned segments of DNA.

The displayed protein does not need to be a fusion protein. For example, a fluorescent binding ligand may attach to a coat protein by virtue of a non-covalent interaction, e.g., a coiled coil binding interaction, such as jun/fos binding, or a covalent interaction mediated by cysteines (see, e.g., Crameri et al., 1994, Eur. J. Biochem., 226:53-58) with or without additional non-covalent interactions. Morphosys have described a display system in which one cysteine is put at the C terminus of the scFv or Fab, and another is put at the N terminus of g3p. The two assemble in the periplasm and display occurs without a fusion gene or protein. A modification of the coiled coil approach was used by Paschke et al., ⁵⁶ to display a circular permutant of GFP. However, that GFP was not fluorescent.

The coat protein does not need to be endogenous. For example, DNA binding proteins can be incorporated into the phage/phagemid genome (see, e.g., McGregor & Robins, 2001, Anal. Biochem., 294:108-117,). When the sequence recognized by such proteins is also present in the genome, the DNA binding protein becomes incorporated into the phage/phagemid. This can serve as a display vector protein. In some cases it has been shown that incorporation of DNA binding proteins into the phage coat can occur independently of the presence of the recognized DNA signal.

Other phage can also be used. For example, T7 vectors, T4 vector, T2 vectors, or lambda vectors can be employed in which the displayed product on the mature phage particle is released by cell lysis.

Another methodology is selectively infective phage (SIP) technology. which provides for the in vivo selection of interacting protein-ligand pairs. A “selectively infective phage” consists of two independent components. For example, a recombinant filamentous phage particle is made non-infective by replacing its N-terminal domains of gene 3 protein (g3p) with a protein of interest, e.g., an antigen. The nucleic acid encoding the antigen can be inserted such that it will be expressed. The second component is an “adapter” molecule in which the fluorescent ligand is linked to those N-terminal domains of g3p that are missing from the phage particle. Infectivity is restored when the displayed protein (e.g., a fluorescent binding ligand) binds to the antigen. This interaction attaches the missing N-terminal domains of g3p to the phage display particle. Phage propagation becomes strictly dependent on the protein-ligand interaction. See, e.g., Spada, 1997, J. Biol. Chem. 378:445-456; Pedrazzi, 1997, FEBS Lett. 415:289-293; Hennecke, 1998, Protein Eng. 11:405-410.

### Other Display Libraries:

In addition to phage display libraries, analogous epitope display libraries can also be used. For example, the methods of the invention can also use yeast surface displayed libraries (see, e.g., Boder, 1997, Nat. Biotechnol., 15:553-557), which can be constructed using such vectors as the pYD1 yeast expression vector. Other potential display systems include mammalian display vectors and E. coli libraries.

In vitro display library formats known to those of skill in the art can also be used, e.g., ribosome displays libraries and mRNA display libraries. In these in vitro selection technologies, proteins are made using cell-free translation and physically linked to their encoding mRNA after in vitro translation. In typical methodology for generating these libraries, DNA encoding the sequences to be selected are transcribed in vitro and translated in a cell-free system.

In ribosome display libraries (see, e.g., Mattheakis et al., 1994, Proc. Natl. Acad. Sci. USA 91:9022-9026; Hanes & Pluckthrun, 1997, Proc. Natl. Acad. Sci. USA, 94:4937-4942) the link between the mRNA encoding the fluorobody of the invention and the ligand is the ribosome itself. The DNA construct is designed so that no stop codon is included in the transcribed mRNA. Thus, the translating ribosome stalls at the end of the mRNA and the encoded protein is not released. The encoded protein can fold into its correct structure while attached to the ribosome. The complex of mRNA, ribosome and protein is then directly used for selection against an immobilized target. The mRNA from bound ribosomal complexes is recovered by dissociation of the complexes with EDTA and amplified by RT-PCR.

Method and libraries based on mRNA display technology, also referred to herein as puromycin display, are described, for example in U.S. Pat. Nos. 6,261,804; 6,281,223; 6,207,446; and 6,214,553. In this technology, a DNA linker attached to puromycin is first fused to the 3′ end of mRNA. The protein is then translated in vitro and the ribosome stalls at the RNA-DNA junction. The puromycin, which mimics aminoacyl tRNA, enters the ribosomal A site and accepts the nascent polypeptide. The translated protein is thus covalently linked to its encoding mRNA. The fused molecules can then be purified and screened for binding activity. The nucleic acid sequences encoding ligands with binding activity can then be obtained, for example, using RT-PCR.

The fluorobody and sequences, e.g., DNA linker for conjugation to puromycin, can be joined by methods well known to those of skill in the art and are described, for example, in U.S. Pat. Nos. 6,261,804; 6,281,223; 6,207,446; and 6,214,553.

Other technologies involve the use of viral proteins (e.g., protein A) that covalently attach themselves to the genes that encodes them. Fusion proteins are created that join the fluorobody to the protein A sequence, thereby providing a mechanism to attach the binding ligands to the genes that encode them.

Plasmid display systems rely on the fusion of displayed proteins to DNA binding proteins, such as the lac repressor (see, e.g., Gates et al., 1996, J. Mol. Biol., 255:373-386; 1996, Methods Enzymol. 267:171-191). When the lac operator is present in the plasmid as well, the DNA binding protein binds to it and can be co-purified with the plasmid. Libraries can be created linked to the DNA binding protein, and screened upon lysis of the bacteria. The desired plasmid/proteins are rescued by transfection, or amplification.

### Screening Libraries:

Advantages inherent to fluorobodies will enable greatly simplified screening procedures. Fluorobodies may be easily tracked through selection and screening steps, both in terms of functionality and expression, by visual inspection of precipitates, solutions, clones and the like. Such visual tracking is not possible with any other known binding ligand scaffold. The ability to visually track fluorobodies using their intrinsic fluorescence provides a unique advantage over standard methodologies, thereby enabling efficient high-throughput strategies for selecting clones. Accordingly, antigen screening of fluorobody libraries may be conducted by monitoring intrinsic fluorescence alone.

Methods of screening the libraries of the invention are well known to those in the art. The libraries are typically screened using an antigen, or molecule of interest, for which it is desirable to select a binding partner. Typically, the antigen is attached to a solid surface or a specific tag, such as biotin. The antigen (or molecule of interest) is incubated with a library of the invention. Those polypeptides that bind to the antigen are then separated from those that do not using any of a number of different methods. These methods involve washing steps, followed by elution steps. Washing can be done, for example, with PBS, or detergent-containing buffers. Elution can be performed with a number of agents, depending on the type of library. For example, an acid, a base, bacteria, or a protease can be used when the library is a phage display library.

To facilitate the identification and isolation of the antigen-bound fluorobody, the ligand can also be engineered as a fusion protein to include selection markers (e.g., epitope tags). Antibodies reactive with the selection tags present in the fusion proteins or moieties that bind to the labels can then be used to isolate the antigen-fluorobody complex via the epitope or label. For example, fluorobody/antigen complexes can be separated from non-complexed display particles using antibodies specific for the antibody selection “tag” e.g., an SV5 antibody specific to an SV5 tag. In libraries that are constructed using a display vector, such as a phage display vector, the selected clones, e.g., phage, are then used to infect bacteria.

Other detection and purification facilitating domains include, e.g., metal chelating peptides such as polyhistidine tracts and histidine-tryptophan modules that allow purification on immobilized metals, protein A domains that allow purification on immobilized immunoglobulin, or the domain utilized in the FLAGS extension/affinity purification system (Immunex Corp, Seattle Wash.). Any epitope with a corresponding high affinity antibody can be used, e.g., a myc tag (see, e.g., Kieke, 1997, Protein Eng. 10:1303-1310) or an E-tag (Pharmacia). See also Maier, 1998, Anal. Biochem. 259:68-73; Muller, 1998, Anal. Biochem. 259:54-61. The inclusion of a cleavable linker sequences such as Factor Xa or enterokinase (Invitrogen, San Diego Calif.) between the purification domain and binding site may be useful to facilitate purification. For example, an expression vector of the invention includes a polypeptide-encoding nucleic acid sequence linked to six histidine residues. A widely used tags is six consecutive histidine residues or 6H is tag. These residues bind with high affinity to metal ions immobilized on chelating resins even in the presence of denaturing agents and can be mildly eluted with imidazole. Another exemplary epitope tag is the Selection tags can also make the epitope or binding partner (e.g., antibody) detectable or easily isolated by incorporation of, e.g., predetermined polypeptide epitopes recognized by a secondary reporter/binding molecule, e.g., leucine zipper pair sequences; binding sites for secondary antibodies; transcriptional activator polypeptides; and other selection tag binding compositions. See also, e.g., Williams, 1995, Biochemistry, 34:1787-1797.

The screening protocols typically employ multiple rounds of selection to identify a binding ligand with the desired properties. For example, it may be desirable to select fluorobodies with a minimum binding avidity for a target. Alternatively, a maximum binding avidity of a target may be desirable. In other uses, it may be desirable to select a fluorobody that is thermostable at a particular temperature. For example, selection using increasingly stringent binding conditions can be used to select binding ligands that bind to a target molecule at increasingly greater binding affinities. One method of performing this selection is by decreasing concentrations of an antigen to select fluorobodies from a library that have a higher affinity for the antigen. A variety of other parameters can also be adjusted to select for high affinity binding ligands, e.g., increasing salt concentration, temperature, and the like. In one embodiment, affinity selection is carried out with FACS, taking advantage of the intrinsic fluorescence of fluorobodies.

Once a fluorobody is selected, the nucleic acid encoding it is readily obtained. This sequence may then be expressed using any of a number of systems to obtain the desired quantities of the protein. There are many expression systems for that are well know to those of ordinary skill in the art. (See, e.g., Gene Expression Systems, Fernandes and Hoeffler, Eds. Academic Press, 1999; Ausubel, supra.) Typically, the polynucleotide that encodes the fluorobody is placed under the control of a promoter that is functional in the desired host cell. An extremely wide variety of promoters are available, and can be used in the expression vectors of the invention, depending on the particular application. Ordinarily, the promoter selected depends upon the cell in which the promoter is to be active. Other expression control sequences such as ribosome binding sites, transcription termination sites and the like are also optionally included. Constructs that include one or more of these control sequences are termed “expression cassettes.” Accordingly, the nucleic acids that encode the joined polypeptides are incorporated for high level expression in a desired host cell.

### Generation of Fluorobodies Specific for Epitopes Recognized by Known Monoclonal Antibodies:

In another aspect of the invention, monoclonal antibodies may be employed to select fluorobodies recognizing the same epitope recognized by the monoclonal antibody. This may be particularly useful, for example, in the generation of fluorobodies having binding properties similar or essentially identical to a particular monoclonal antibody. In this regard, a large number of highly specific monoclonal antibodies are widely used in molecular medicine for diagnostic and other purposes, as well as in a broad range of biomedical research and drug discovery contexts. The invention provides a means for generating functionally equivalent fluorobodies that may be used in place of such monoclonal antibodies for the same purposes, thus replacing a binding ligand which requires the use of secondary detection agents with one that has intrinsic fluorescence and thus instant detectability.

In one embodiment, a fluorobody library is screened against an antigen of interest. Fluorobodies specific for the epitope recognized by the monoclonal antibody of interest may be selected by the addition of an excess of the monoclonal antibody sufficient to elute fluorobodies occupying (or bound to) the same epitope. Additional rounds of selection may be desirable. The resulting selected fluorobody would be expected to be immunologically identical to the monoclonal antibody in virtually all diagnostic, imaging, screening, immunoassay, etc. contexts.

In a specific embodiment, a method for generating a fluorobody recognizing a specific epitope of an antigen comprises the steps of (a) screening a fluorobody library with the antigen of interest, and selecting clones which bind to the antigen, (b) re-binding the selected clones to the antigen, (c) contacting the antigen-bound clones with an excess quantity of a monoclonal antibody which binds specifically to the epitope, such quantity to be sufficient to elute any clones bound to antigen via the same epitope, and (d) selecting the eluted clones. Optionally, the method further comprises re-binding the eluted clones to the antigen, followed by elution of epitope-specific clones with the monoclonal antibody.

### Use of Fluorobodies:

The fluorobodies of the invention will be useful in a large range of applications currently employing antibodies and antibody derivatives, as will be readily appreciated by those skilled in the art. For example, fluorobodies will be useful in essentially all research, diagnostic, assay, and imaging contexts in which polyclonal and monoclonal antibodies (and related molecules) have been used for many years, including without limitation, in standard immunoassays such as ELISA, immunoprecipitation, immunohistochemistry, immunoblot and the like.

Fluorobodies will also be useful as affinity reagents for the isolation, separation, and purification of proteins, and as detection reagents in protein arrays. Other uses include fluorobody biosensors and fluorobody imaging. Fluorobodies will also be useful in a variety of other research contexts, including the study of protein-protein interaction utilizing fluorobodies capable of identifying protein interactions via FRET (see Tsien et al, 1998, supra; Pollok and Heim, 1999, Trends Cell Biol. 9:57-60; Margolin et al., 2000, Methods 20: 62-72). In all contexts, fluorobodies will provide distinct advantages over traditionally utilized antibody and antibody derivative reagents due to their inherent fluorescence, thereby eliminating the need for secondary detection reagents, as well as due to their high stability and other desirable advantages.

Fluorobodies will also find use in various in vivo diagnostic and imaging applications. In such applications utilizing fluorobodies, the use of far-red, preferably near infrared emission variants may be preferred, as these wavelengths are best able to penetrate through live tissue. The use of such fluorobodies may be particularly desirable in whole body imaging, tumor localization imaging, etc.

In one embodiment of this aspect of the invention, the fluorescence of superficial structures to which fluorobodies are bound may be imaged in vivo using confocal or multiphoton microscopy (see, Brown et al., 2001, Nature Med. 7: 864-868).

In another embodiment, diseased body tissues may be detected using fluorobodies specific for proteins contained within or expressed on the surface of cells within the tissue of interest. Preferably, such proteins are unique to or preferentially over-expressed in the disease state of the tissue relative to normal. In a typical application, fluorobodies specific for a tumor antigen may be used to image the tumor tissue in vivo. Depending upon the nature of the imaging problem presented, fluorobodies may be administered directly onto the tissue or organ of interest in order to facilitate the binding of the fluorobody to the target tissue. In other applications, it may be desirable to inject the fluorobodies intravenously, such as in situations where visualization of metastatic lesions as well as the primary tumor are of interest.

Fluorescence is detected following excitation with the appropriate wavelength of light as is well known in the art, including for example, visualization by a CCD camera. The angles at which excitation light irradiation of the target tissue is presented will vary depending upon the anatomical context of the target tissue, as will the angle at which light emission is detected.

A further particular embodiment relates to the use of fluorobodies in fluorescence molecular tomography (FMT). FMT is a recently described volumeric imaging technology which accounts for the diffusive propagation of photons in living tissues (Ntziachristos et al., 2002, Nature Med. 8: 757-760). FMT using enzyme-activatable fluorochromes detected with near infrared light has been used to image brain tumors in mice. This technology may be extended to using the fluorobodies of the invention. More specifically, tumors may be imaged using fluorobodies specific for tumor-specific markers. In this regard, a great number of cell surface tumor markers have been identified, and these may be used to screen fluorobody libraries for monoclonal fluorobodies highly specific for such tumor markers. These tumor-specific fluorobodies may be introduced in vivo and the location and volume of the target tumor tissue determined using FMT. The use of fluorobodies in this application will enable precise localization and monitoring over time.

Fluorobody imaging of tumors is expected to be enormously useful in the diagnosis, monitoring and treatment of patients with cancer. In particular, the precise localization of tumors not only provides diagnostic and prognostic information, but also may revolutionize the precision with which tumors can be surgically removed. Even in situations where tumors are easily localized using existing imaging methodologies, the surgical excision of such tumors typically involves the removal of significant sections of normal tissue from the patient, resulting from the conservative definition of surgical margins necessitated by the difficulty in visually determining precisely where the tumor tissue ends. The use of tumor-specific fluorobodies may enable the real-time precise imaging of tumor tissue in the operating room, enabling surgeons to better and more precisely visualize the tumor tissue in need of excision, as well as any infiltrated lymph nodes or metastatic lesions in need of excision as well. In one application of this aspect of the invention, far-red or preferably near infrared emission spectra fluorobodies capable of specifically binding to a tumor antigen are used, in order to take advantage of the ability of far-red light to propagate through tissue more than other light wavelengths (see Ntziachristos et al., supra).

The stability of fluorobodies will enable the emission of detectable fluorescence from the target tumor tissue for hours without loss of fluorescence. This feature may be particularly useful in the surgical excision of diffuse margin tumors, which may take many hours of painstaking surgery. Indeed, some tumors are so diffuse that a clinical decision not to attempt surgical removal is frequently made. For example, the brain neoplasm glioblastoma grows in tentacle-like fashion, and the margins of glioblastoma cannot be sufficiently localized to indicate or guide effective surgical removal. Accordingly, glioblastoma is often considered a terminal condition precluding surgical therapeutic intervention. The use of a fluorobody specific for a glioblastoma cell surface antigen, for example, would enable direct visualization of the tumor margins, perhaps enabling effective surgical removal of glioblastoma tissue from the patient's brain. In other oncology applications, fluorobody stability over a wide pH range may facilitate their detection, when fluorobodies are directed to targets which undergo internalization and as a result are directed to the acidic phagolysozomal compartment of the tumor cells.

Fusion constructs of GFP or other fluorescent proteins and human antibodies or single chain antibodies are incapable of accessing the interior of a cell without the further addition of cell localization signal peptides Such chimeras are large molecules with variable stabilities. Fluorobodies, on the other hand, are vastly more stable and considerably smaller, permitting their potential use as self-directing intracellular markers.

For use in the diagnostic applications described or suggested above, kits are also provided by the invention. Such kits may comprise a carrier means being compartmentalized to receive in close confinement one or more container means such as vials, tubes, and the like, each of the container means comprising one of the separate elements to be used in the method. For example, one of the container means may comprise a fluorobody specific for a protein or antigen of interest.

Various therapeutic uses are also contemplated. Fluorobodies may be used therapeutically, much in the same manner that antibodies and antibody derivatives have been used. In one general embodiment, for example, therapeutic drugs or isotopes may be conjugated to a fluorobody using standard techniques and administered to their targets.

In one embodiment, fluorobodies may be used to treat cancer. Fluorobodies specifically reactive with cell-surface tumor antigens may be useful to treat cancer systemically, either as toxin or therapeutic agent conjugates or, potentially, as unconjugated fluorobodies capable of inhibiting cell proliferation or function.

Fluorobodies specific for a tumor antigen may be introduced into a patient such that the fluorobody binds to the tumor antigen on or in the cancer cells and thereby mediates the destruction of the cells and the tumor and/or inhibits the growth of the cells or the tumor. Mechanisms by which such fluorobodies exert a therapeutic effect may include modulating the physiologic function of the tumor antigen, inhibiting ligand binding or signal transduction pathways, modulating tumor cell differentiation, altering tumor angiogenesis factor profiles, and/or by inducing apoptosis. Anti-tumor fluorobodies conjugated to toxic or therapeutic agents may also be used therapeutically to deliver the toxic or therapeutic agent directly to antigen-bearing tumor cells.

Fluorobodies are likely to be cleared rapidly from the circulation, due to their relatively small size, which is below the renal threshold. Extrapolating from experiments with antibody fragments (scFvs, Fabs, minibodies, scFv dimers), it is clear that the circulation clearance time can be increased by increasing the mass of the fluorobody. This may be done by those with skill in the art by dimerization, and/or by the addition of a large tag, such as an antibody constant domain, or domains.

Therapeutic fluorobodies may be formulated into pharmaceutical compositions comprising a carrier suitable for the desired delivery method. Suitable carriers include any material which when combined with the fluorobody retains their anti-tumor function and is nonreactive with the subject's immune system. Examples include, but are not limited to, any of a number of standard pharmaceutical carriers such as sterile phosphate buffered saline solutions, bacteriostatic water, and the like.

Therapeutic fluorobody formulations may be administered via any route capable of delivering them to the tumor site. Potentially effective routes of administration include, but are not limited to, intravenous, intraperitoneal, intramuscular, intratumor, intradermal, and the like. The preferred route of administration is by intravenous injection. A preferred formulation for intravenous injection comprises the fluorobody in a solution of preserved bacteriostatic water, sterile unpreserved water, and/or diluted in polyvinylchloride or polyethylene bags containing 0.9% sterile Sodium Chloride for Injection, USP. The preparation may be lyophilized and stored as a sterile powder, preferably under vacuum, and then reconstituted in bacteriostatic water containing, for example, benzyl alcohol preservative, or in sterile water prior to injection.

Depending upon the nature and location of the therapeutic target, the use of fluorobodies in such therapeutic application may also enable the tracking of the therapeutic composition within the patient and, ultimately, to the target(s).

### EXAMPLES

### Example 1

### Generation of a Fluorobody Library in a Cytoplasmic Expression Vector

### Materials and Methods:

With reference to FIG. 2, the following TGP loops are selected for the insertion of diversity via a restricted set of random peptides comprising at least three amino acids, tyrosine, glycine and serine: Loop 2: 96/97, loop 3: 164/165 and loop 6: 124-135 (referencing FIG. 2, in the following order: loops 2, 6, 3 as underlined). In loop 2 the library size ranges from 3-13 randomized amino acids, with the smallest library (3 aa) being replacement of three amino acids, and the largest library (13 aa), being replacement of three and addition of another 10 amino acids. In the case of loop 6, it is only replacement of the highlighted seven amino acids with the replacement amino acids comprising tyrosine, glycine or serine, as well as the original amino acid found at each replacement position. In the case of loop 3, the smallest library (7 aa) has seven amino acids replaced, while the longest (14 aa) has seven aa replaced and seven inserted.

A pET based vector, pETCK3, was used as the cloning vehicle to create initial libraries. In a first step, the loop 6 primer 3 oligonucleotide (see Table of Sequences, infra) was used to create a single loop library in TGP. This oligo introduced changes at 7 sites in loop 6. The changes are replacements in which the natural amino acids are replaced with tyrosine, serine, glycine, or the original amino acid. 160 highly fluorescent clones were picked and sequenced. These were diverse and used as a substrate for the next phase of the library creation. In parallel, all the loop 2 primers and all the loop 3 primers were used to create additional single loop libraries on unmodified TGP. The final library was created by amplifying the library portions of the loop 2, loop 3 and loop 6 libraries, and assembling them into a single triple loop library using assembly PCR.

The full length gene was, then amplified with primers:

CGP-Hind3-3′:
TTTGCCAAGCTTGCGGCTAGCTTTAGCCTGAGACGGTAAC
CGP-EcoRI-5′:
TACATATGAATTCGGGCGCGCATGCCTCAGTAATTAAAC

that were compatible with Novagen's T7Select 10⁻³b cloning vector. The amplicon was digested with EcoRI/HindIII and ligated into the T7 vector arms. The packaging reaction was performed according to the manufacturer's recommendations.

The resulting triple loop library was titrated and found to have a final diversity of 3×10⁸ pfu/ml. The library was amplified and 10¹⁰ virus particles were used for each selection against lysozyme. The biopanning was performed on a Kingfisher purification system with biotinylated antigen. After each round of selection 100 μl of fresh BLT5403 cells were infected and the selected virus population was amplified for 8 h at 25° C. The biopanning process became more and more stringent after each round. The resulting virus particles were collected after the 3^{rd} round of selection and the fluorobody inserts were amplified with primers compatible with the pETCK3 bacterial expression vector:

CGP-BssHII-5′:
[SEQ ID NO: 33]
TACATATGGGCGCGCATGCCTCAGTAATTAAACCG
CGP-NheI-3′:
[SEQ ID NO: 34]
TTTGCCGCTAGCTTTAGCCTGAGACGGTAACATAGAATAGC

The PCR product was digested with BssHII/NheI and inserted into the expression vector. After transfection into BL21(DE3)-Gold cells the green colonies were picked and analyzed by ELISA and FLISA for their binding specificity to the antigen that they were selected against. Three clones showed positivity against the lysozyme target.

### Results:

Several putative anti-lysozyme fluorobodies were obtained, and evaluated against the antigen against which they were selected for binding performance in a standard ELISA, along with two irrelevant antigens (myoglobin and neutravidin). The results are presented in FIG. 7, and show specific binding of fluorobodies C10 and C12 to lysozyme relative to the irrelevant antigens.

### Example 2

### Generation of Non-Aggregating eCGP123 Fluorobody Scaffolds

In order to eliminate aggregation tendencies encountered with the eCGP123 fluorobody scaffold, various structural modifications to eCGP123 were designed and tested. Three eCGP variants were generated. In the first, the last seven amino acids of the eCGP structure were replaced with the sequence gly-gly-gly-ser-gly-gly-gly [SEQ ID NO: 5]. In the second and third [SEQ ID NOS: 7 and 9], the negative charge of eCGP123 was increased by including more negatively amino acids. The three improved eCGP proteins, termed nsTGP(−1), nsTGP(−10) and nsTGP(−18) were expressed and evaluated for aggregation tendencies, in comparison to eCGP. All three variants showed no aggregation after dialysis into PBS and exhibited very similar thermostability.

All publications, patents, and patent applications cited in this specification are herein incorporated by reference as if each individual publication or patent application were specifically and individually indicated to be incorporated by reference.

The present invention is not to be limited in scope by the embodiments disclosed herein, which are intended as single illustrations of individual aspects of the invention, and any which are functionally equivalent are within the scope of the invention. Various modifications to the models and methods of the invention, in addition to those described herein, will become apparent to those skilled in the art from the foregoing description and teachings, and are similarly intended to fall within the scope of the invention. Such modifications or other embodiments can be practiced without departing from the true scope and spirit of the invention.

TABLE OF SEQUENCES
SEQ ID NO: 1 - eCGP123 amino acid sequence
MSVIKPEMKIKLRMEGAVNGHKFVIEGEGIGKPYEGTQTLDLTVKEGAPLPFSYDILT
PAFQYGNRAFTKYPKDIPDYFKQAFPEGYSWERSMTYEDQGICIATSDITMEGDCF
FYKIRFDGTNFPPNGPVMQKKTLKWEPSTEKMYVRDGVLKGDVNMALLLEGGGHY
RCDFKTTYKAKKDVRLPDAHEVDHRIEILSHDKDYNKVRLYEHAEARYSMLPSQAK
SEQ ID NO: 2 - eCGP123 amino acid sequence
ATGTCAGTAATTAAACCGGAAATGAAAATTAAATTGCGTATGGAAGGTGCCGTTAACGG
CCATAAATTTGTAATTGAAGGAGAAGGAATAGGCAAACCATACGAAGGAACCCAGACC
CTGGATTTAACCGTAAAAGAAGGCGCACCTCTCCCTTTCTCGTACGACATCCTCACCC
CAGCCTTCCAATACGGCAATCGCGCTTTCACCAAATACCCAAAAGATATTCCAGACTAT
TTTAAACAAGCATTCCCCGAAGGCTATTCTTGGGAACGCTCTATGACCTATGAAGATCA
AGGAATTTGTATCGCTACCTCCGACATTACTATGGAAGGAGACTGTTTTTTTTATAAGAT
TCGCTTTGATGGAACTAACTTCCCCCCGAACGGCCCTGTAATGCAAAAGAAGACCTTA
AAATGGGAACCTAGCACCGAAAAAATGTATGTACGCGACGGAGTTCTTAAGGGTGACG
TAAACATGGCACTTCTGCTCGAAGGAGGTGGACACTACCGCTGCGATTTTAAAACCAC
TTATAAAGCCAAAAAAGATGTTCGTCTTCCAGATGCACACGAGGTGGACCACCGCATT
GAAATCCTGAGCCACGATAAAGATTATAATAAAGTTAGACTCTATGAACACGCCGAAGC
CCGCTATTCTATGTTACCGTCTCAGGCTAAAGCTAGC
SEQ ID NO: 3 - TGP amino acid sequence
(difference relative to eCGP sequence shown in bold underline)
MSVIKPEMKIKLRMEGAVNGHKFVIEGEGIGKPYEGTQTLDLTVKEGAPLPFSYDILT
PAFQYGNRAFTKYPKDIPDYFKQAFPEGYSWERSMTYEDQGICIATSDITMEGDCF
FYKIRFDGTNFPPNGPVMQKKTLKWEPSTEKMYVRDGVLKGDVNMALLLEGGGHY
RCDFKTTYKAKKDVRLPDAHKVDHRIEILSHDKDYNKVRLYEHAEARYSMLPSQAK
SEQ ID NO: 4 - TGP nucleotide coding sequence
(difference relative to eCGP sequence shown in bold underline)
GCGCGCATGCCTCAGTAATTAAACCGGAAATGAAAATTAAATTGCGTATGGAAG
GTGCCGTTAACGGCCATAAATTTGTAATTGAAGGAGAAGGAATAGGCAAACCAT
ACGAAGGAACCCAGACCCTGGATTTAACCGTAAAAGAAGGCGCACCTCTCCCT
TTCTCGTACGACATCCTCACCCCAGCCTTCCAATACGGCAATCGCGCTTTCACC
AAATACCCAAAAGATATTCCAGACTATTTTAAACAAGCATTCCCCGAAGGCTATT
CTTGGGAACGCTCTATGACCTATGAAGATCAAGGAATTTGTATCGCTACCTCCG
ACATTACTATGGAAGGAGACTGTTTTTTTTATAAGATTCGCTTTGATGGAACTAA
CTTCCCCCCGAACGGCCCTGTAATGCAAAAGAAGACCTTAAAATGGGAACCTAG
CACCGAAAAAATGTATGTACGCGACGGAGTTCTTAAGGGTGACGTAAACATGGC
ACTTCTGCTCGAAGGAGGTGGACACTACCGCTGCGATTTTAAAACCACTTATAA
AGCCAAAAAAGATGTTCGTCTTCCAGATGCACACAAAGTGGACCACCGCATTGA
AATCCTGAGCCACGATAAAGATTATAATAAAGTTAGACTCTATGAACACGCCGAA
GCCCGCTATTCTATGTTACCGTCTCAGGCTAAAGCTAGC
SEQ ID NO: 5 - nsTGP(−1) nucleotide sequence
TCAGTAATTAAACCGGAAATGAAAATTAAATTGCGTATGGAAGGTGCCGTTAAC
GGCCATAAATTTGTAATTGAAGGAGAAGGAATAGGCAAACCATACGAAGGAACC
CAGACCCTGGATTTAACCGTAAAAGAAGGCGCACCTCTCCCTTTCTCGTACGAC
ATCCTCACCCCAGCCTTCCAATACGGCAATCGCGCTTTCACCAAATACCCAAAA
GATATTCCAGACTATTTTAAACAAGCATTCCCCGAAGGCTATTCTTGGGAACGCT
CTATGACCTATGAAGATCAAGGAATTTGTATCGCTACCTCCGACATTACTATGGA
AGGAGACTGTTTTTTTTATAAGATTCGCTTTGATGGAACTAACTTCCCCCCGAAC
GGCCCTGTAATGCAAAAGAAGACCTTAAAATGGGAACCTAGCACCGAAAAAATG
TATGTACGCGACGGAGTTCTTAAGGGTGACGTAAACATGGCACTTCTGCTCGAA
GGAGGTGGACACTACCGCTGCGATTTTAAAACCACTTATAAAGCCAAAAAAGAT
GTTCGTCTTCCAGATGCACACGAGGTGGACCACCGCATTGAAATCCTGAGCCA
CGATAAAGATTATAATAAAGTTAGACTCTATGAACACGCCGAAGCCCGCTATTCT
ggcggaggcagcggaggcggt
SEQ ID NO: 6 - nsTGP(−1) amino acid sequence
AHASVIKPEMKIKLRMEGAVNGHKFVIEGEGIGKPYEGTQTLDLTVKEGAPLPFSYDI
LTPAFQYGNRAFTKYPKDIPDYFKQAFPEGYSWERSMTYEDQGICIATSDITMEGD
CFFYKIRFDGTNFPPNGPVMQKKTLKWEPSTEKMYVRDGVLKGDVNMALLLEGGG
HYRCDFKTTYKAKKDVRLPDAHEVDHRIEILSHDKDYNKVRLYEHAEARYSGGGSG
GG
SEQ ID NO: 7 - nsTGP(−10) nucleotide sequence
TCAGTAATTAAACCGGAAATGAAAATTAAATTGCGTATGGAAGGTGCCGTTAAC
GGCCATAAATTTGTAATTGAAGGAGAAGGAATAGGCAAACCATACGAAGGAACC
CAGACCCTGGATTTAACCGTAGAAGAAGGCGCACCTCTCCCTTTCTCGTACGAC
ATCCTCACCCCAGCCTTCCAATACGGCAATCGCGCTTTCACCAAATACCCAGAA
GATATTCCAGACTATTTTAAACAAGCATTCCCCGAAGGCTATTCTTGGGAACGCT
CTATGACCTATGAAGATCAAGGAATTTGTATCGCTACCTCCGACATTACTATGGA
AGGAGACTGTTTTTTTTATGAAATTCGCTTTGATGGAACTAACTTCCCCCCGAAC
GGCCCTGTAATGCAAAAGAAGACCTTAAAATGGGAACCTAGCACCGAAAAAATG
TATGTAGAAGACGGAGTTCTTAAGGGTGACGTAGAAATGGCACTTCTGCTCGAA
GGAGGTGGACACTACCGCTGCGATTTTAAAACCACTTATAAAGCCAAAAAAGAT
GTTCGTCTTCCAGATGCACACGAGGTGGACCACCGCATTGAAATCCTGAGCCA
CGATAAAGATTATAATAAAGTTAGACTCTATGAACACGCCGAAGCCCGCTATTCT
ggcggaggcagcggaggcggt
SEQ ID NO: 8 - nsTGP(−10) amino acid sequence
SVIKPEMKIKLRMEGAVNGHKFVIEGEGIGKPYEGTQTLDLTVEEGAPLPFSYDILTP
AFQYGNRAFTKYPEDIPDYFKAQAFPEGYSWERSMTYEDQGICIATSDITMEGDCFF
YEIRFDGTNFPPNGPVMQKKTLKWEPSTEKMYVEDGVLKGDVEMALLLEGGGHYR
CDFKTTYKAKKDVRLPDAHEVDHRIEILSHDKDYNKVRLYEHAEARYSGGGSGGG
SEQ ID NO: 9 - nsTGP(−18) nucleotide sequence
TCAGTAATTGAACCGGAAATGAAAATTAAATTGCGTATGGAAGGTGCCGTTAAC
GGCCATAAATTTGTAATTGAAGGAGAAGGAATAGGCAAACCATACGAAGGAACC
CAGACCCTGGATTTAACCGTAGAAGAAGGCGCACCTCTCCCTTTCTCGTACGAC
ATCCTCACCCCAGCCTTCCAATACGGCAATCGCGCTTTCACCGAATACCCAGAA
GATATTCCAGACTATTTTAAACAAGCATTCCCCGAAGGCTATTCTTGGGAACGCT
CTATGACCTATGAAGATCAAGGAATTTGTATCGCTACCTCCGACATTACTATGGA
AGGAGACTGTTTTTTTTATGAAATTGAATTTGATGGAACTAACTTCCCCCCGAAC
GGCCCTGTAATGCAAAAGAAGACCTTAAAATGGGAACCTAGCACCGAAAAAATG
TATGTAGAAGACGGAGTTCTTAAGGGTGACGTAGAAATGGCACTTCTGCTCGAA
GGAGGTGGACACTACCGCTGCGATTTTAAAACCACTTATAAAGCCAAAAAAGAT
GTTCGTCTTCCAGATGCACACGAGGTGGACCACGAAATTGAAATCCTGAGCCA
CGATAAAGATTATAATAAAGTTAGACTCTATGAACACGCCGAAGCCCGCTATTCT
ggcggaggcagcggaggcggt
SEQ ID NO: 10 - nsTGP(−18) amino acid sequence
SVIEPEMKIKLRMEGAVNGHKFVIEGEGIGKPYEGTQTLDLTVEEGAPLPFSYDILTP
AFQYGNRAFTEYPEDIPDYFKQAFPEGYSWERSMTYEDQGICIATSKITMEGDCFF
TEIEFDGTNFPPNGPVMQKKTLKWEPSTEKMYVEDGVLKGDVEMALLLEGGGHYR
CDFKTTYKAKKDVRLPDAHEVDHEIEILSHDKDYNKVRLYEHAEARYSGGGSGGG
OLIGONUCLEOTIDES USED TO CREATE YSG LIBRARIES:
X in the sequences below corresponds to a trimer mix comprising
codons encoding 50% tyrosine, 25% glycine and 25% serine.
Individual oligo/total diversity for each series indicated right
of the sequences.
LOOP 2 PRIMERS:
lp2-GSY3-5
[SEQ ID NO: 11]
GGGAACGCTCTATGACCTATXXXGGAATTTGTATCGCTACCTCC - 27
lp2-GSY4-5
[SEQ ID NO: 12]
GGGAACGCTCTATGACCTATXXXXGGAATTTGTATCGCTACCTCC - 81/108
lp2-GSY5-5
[SEQ ID NO: 13]
GGGAACGCTCTATGACCTATXXXXXGGAATTTGTATCGCTACCTCC - 243/351
lp2-GSY6-5
[SEQ ID NO: 14]
GGGAACGCTCTATGACCTATXXXXXXGGAATTTGTATCGCTACCTCC - 729/1080
lp2-GSY7-5
[SEQ ID NO: 15]
GGGAACGCTCTATGACCTATXXXXXXXGGAATTTGTATCGCTACCTCC - 2,187/3,269
lp2-GSY8-5
[SEQ ID NO: 16]
GGGAACGCTCTATGACCTATXXXXXXXXGGAATTTGTATCGCTACCTCC - 6,561/9,830
lp2-GSY9-5
[SEQ ID NO: 17]
GGGAACGCTCTATGACCTATXXXXXXXXXGGAATTTGTATCGCTACCTCC - 19,683/29,513
lp2-GSY10-5
[SEQ ID NO: 18]
GGGAACGCTCTATGACCTATXXXXXXXXXXGGAATTTGTATCGCTACCTCC - 59,049/88,562
lp2-GSY11-5
[SEQ ID NO: 19]
GGGAACGCTCTATGACCTATXXXXXXXXXXXGGAATTTGTATCGCTACCTCC - 177,147 / 265,709
lp2-GSY12-5
[SEQ ID NO: 20]
GGGAACGCTCTATGACCTATXXXXXXXXXXXXGGAATTTGTATCGCTACCTCC - 531,441 / 797,150
lp2-GSY13-5
[SEQ ID NO: 21]
GGGAACGCTCTATGACCTATXXXXXXXXXXXXXGGAATTTGTATCGCTACCTCC - 1,594,323 / 2,391,473
LOOP 3 PRIMERS:
lp3-GSY7-5
[SEQ ID NO: 22]
GGGTGACGTAAACATGGCACTTCTGXXXXXXXCGCTGCGATTTTAAAACC - 2,189
lp3-GSY8-5
[SEQ ID NO: 23]
GGGTGACGTAAACATGGCACTTCTGXXXXXXXXCGCTGCGATTTTAAAACC - 6,561/8,750
lp3-GSY9-5
[SEQ ID NO: 24]
GGGTGACGTAAACATGGCACTTCTGXXXXXXXXXCGCTGCGATTTTAAAACC - 19,683/28,433
lp3-GSY10-5
[SEQ ID NO: 25]
GGGTGACGTAAACATGGCACTTCTGXXXXXXXXXXCGCTGCGATTTTAAAACC - 59,049/87,482
lp3-GSY11-5
[SEQ ID NO: 26]
GGGTGACGTAAACATGGCACTTCTGXXXXXXXXXXXCGCTGCGATTTTAAAACC - 177,147/264,629
lp3-GSY12-5
[SEQ ID NO: 27]
GGGTGACGTAAACATGGCACTTCTGXXXXXXXXXXXXCGCTGCGATTTTAAAACC - 531,441/796,070
lp3-GSY13-5
[SEQ ID NO: 28]
GGGTGACGTAAACATGGCACTTCTGXXXXXXXXXXXXXCGCTGCGATTTTAAAACC - 1,594,323/2,390,393
lp3-GSY14-5
[SEQ ID NO: 29]
GGGTGACGTAAACATGGCACTTCTGXXXXXXXXXXXXXXCGCTGCGATTTTAAAACC - 4,782,969/7,173,362
LOOP 6 PRIMER 1:
[SEQ ID NO: 30]
GATTCGCTTTGATGGAACTXTTCXXXXXGTAATGXAAGXACCTTAAAATGGGAACCTAG - 6,561
LOOP 6 PRIMER 2:
[SEQ ID NO: 31]
GATTCGCTTTGATGGAACT (NGSY) TTC (PGSY) (PGSY) (NGSY) (GSY) (PGSY) GTAATG
(QGSY) AAG (KGSY) ACCTTAAAATGGGAACCTAG - 49,152
LOOP 6 PRIMER 3:
[SEQ ID NO: 32]
GATTCGCTTTGATGGAACT(NGSY)TTC(PGSY)(PGSY)(NGSY)GGC(PGSY)GTAATG
(QGSY)AAG(KGSY)ACCTTAAAATGGGAACCTAG - 16,384
In this case 50% Y,20% G, 20% S and 10% for the additional codon (P, N, Q or K).
In the case of the GSY codon, the ratio should be as before: 50% Y; 25% G and
25% S.

### FOOTNOTE CITED LITERATURE

¹ Smith, G. P. Surface presentation of protein epitopes using bacteriophage expression systems. Curr. Opin. Biotechnol. 2, 668-673 (1991).
² Scott, J. K. & Smith, G. P. Searching for peptide ligands with an epitope library. Science 249, 386-390 (1990).
³ Sblattero, D. & Bradbury, A. Exploiting recombination in single bacteria to make large phage antibody libraries. Nat. Biotechnol. 18, 75-80 (2000).
⁴ Marks, J. D. et al. By-passing immunization. Human antibodies from V-gene libraries displayed on phage. J. Mol. Biol. 222, 581-597 (1991).
⁵ Vaughan, T. J. et al. Human antibodies with sub-nanomolar affinities isolated from a large non-immunised phage display library. Nat. Biotechnol. 14, 309-314 (1996).
⁶ Hufton, S. E. et al. Development and application of cytotoxic T lymphocyte-associated antigen 4 as a protein scaffold for the generation of novel binding ligands. FEBS Lett. 475, 225-231 (2000).
⁷ Schlehuber, S., Beste, G. & Skerra, A. A novel type of receptor protein, based on the lipocalin scaffold, with specificity for digoxigenin. J. Mol. Biol. 297, 1105-1120 (2000).
⁸ Nord, K. et al. Binding proteins selected from combinatorial libraries of an alpha-helical bacterial receptor domain. Nat. Biotechnol. 15, 772-777 (1997).
⁹ van den Beucken, T. et al. Building novel binding ligands to B7.1 and B7.2 based on human antibody single variable light chain domains. J. Mol. Biol. 310, 591-601 (2001).
¹⁰ Davies, J. & Riechmann, L. Single antibody domains as small recognition units: design and in vitro antigen selection of camelized, human VH domains with improved protein stability. Protein Eng. 9, 531-537 (1996).
¹¹ Tsien, R. Y. The green fluorescent protein. Annu Rev Biochem 67, 509-544 (1998).
¹² Shi, H. & Wen Su, W. Display of green fluorescent protein on Escherichia coli cell surface. Enzyme Microb Technol 28, 25-34 (2001).
¹³ Abedi, M. R., Caponigro, G. & Kamb, A. Green fluorescent protein as a scaffold for intracellular presentation of peptides. Nucleic Acids Res. 26, 623-630 (1998).
¹⁴ Peelle, B. et al. Intracellular protein scaffold-mediated display of random peptide libraries for phenotypic screens in mammalian cells. Chem Biol 8, 521-534 (2001).
¹⁵ Zeytun, A., Jeromin, A., Scalettar, B. A., Waldo, G. S. & Bradbury, A. R. Fluorobodies combine GFP fluorescence with the binding characteristics of antibodies. Nat Biotechnol 21, 1473-1479 (2003).
¹⁶ Zeytun, A., Jeromin, A., Scalettar, B. A., Waldo, G. S. & Bradbury, A. R. Retraction. Fluorobodies combine GFP fluorescence with the binding characteristics of antibodies. Nat Biotechnol 22, 601 (2004).
¹⁷ Dai, M. et al. Using T7 phage display to select GFP-based binders. Protein Eng Des Sel 21, 413-424, doi:gzn016 [pii] 10.1093/protein/gzn016 (2008).
Pedelacq, J. D., Cabantous, S., Tran, T., Terwilliger, T. C. & Waldo, G. S. Engineering and characterization of a superfolder green fluorescent protein. Nat Biotechnol 24, 79-88 (2006).
¹⁹ Siegel, M. S. & Isacoff, E. Y. A genetically encoded optical probe of membrane voltage. Neuron 19, 735-741 (1997).
²⁰ Doi, N. & Yanagawa, H. Design of generic biosensors based on green fluorescent proteins with allosteric sites by directed evolution. FEBS Lett. 453, 305-307 (1999).
²¹ Baird, G. S., Zacharias, D. A. & Tsien, R. Y. Circular permutation and receptor insertion within green fluorescent proteins. Proc. Natl. Acad. Sci. U.S.A. 96, 11241-11246 (1999).
²² Miesenbock, G., De Angelis, D. A. & Rothman, J. E. Visualizing secretion and synaptic transmission with pH-sensitive green fluorescent proteins. Nature 394, 192-195 (1998).
²³ Dai, M. et al. The creation of a novel fluorescent protein by guided consensus engineering. Protein Eng Des Sel 20, 69-79 (2007).
²⁴ Karasawa, S., Araki, T., Yamamoto-Nino, M. & Miyawaki, A. A green-emitting fluorescent protein from Galaxeidae coral and its monomeric version for use in fluorescent labeling. J Biol Chem 278, 34167-34171 (2003).
²⁵ Binz, H. K., Stumpp, M. T., Forrer, P., Amstutz, P. & Pluckthun, A. Designing repeat proteins: well-expressed, soluble and stable proteins from combinatorial libraries of consensus ankyrin repeat proteins. J Mol Biol 332, 489-503 (2003).
²⁶ Binz, H. K. et al. High-affinity binders selected from designed ankyrin repeat protein libraries. Nat Biotechnol 22, 575-582 (2004).
²⁷ Davies, J. & Riechmann, L. Antibody VH domains as small recognition units. Biotechnology (N Y) 13, 475-479 (1995).
²⁸ Arbabi Ghahroudi, M., Desmyter, A., Wyns, L., Hamers, R. & Muyldermans, S. Selection and identification of single domain antibody fragments from camel heavy-chain antibodies. FEBS Lett. 414, 521-526 (1997).
²⁹ Nuttall, S. D. et al. Design and expression of soluble CTLA-4 variable domain as a scaffold for the display of functional polypeptides. Proteins 36, 217-227 (1999).
³⁰ Nord, K. et al. Binding proteins selected from combinatorial libraries of an alpha-helical bacterial receptor domain. Nat Biotechnol 15, 772-777 (1997).
³¹ Wikman, M. et al. Selection and characterization of HER2/neu-binding affibody ligands. Protein Eng Des Sel 17, 455-462 (2004).
³² Beste, G., Schmidt, F. S., Stibora, T. & Skerra, A. Small antibody-like proteins with prescribed ligand specificities derived from the lipocalin fold. Proc Natl Acad Sci U S A 96, 1898-1903 (1999).
³³ Vogt, M. & Skerra, A. Construction of an artificial receptor protein (“anticalin”) based on the human apolipoprotein D. Chembiochem 5, 191-199 (2004).
³⁴ Scalley-Kim, M., Minard, P. & Baker, D. Low free energy cost of very long loop insertions in proteins. Protein Sci 12, 197-206 (2003).
³⁵ Minard, P., Scalley-Kim, M., Watters, A. & Baker, D. A “loop entropy reduction” phage-display selection for folded amino acid sequences. Protein Sci 10, 129-134 (2001).
³⁶ Bessette, P. H., Rice, J. J. & Daugherty, P. S. Rapid isolation of high-affinity protein binding peptides using bacterial display. Protein Eng Des Sel 17, 731-739 (2004).
³⁷ Camaj, P., Hirsh, A. E., Schmidt, W., Meinke, A. & von Gabain, A. Ligand-mediated protection against phage lysis as a positive selection strategy for the enrichment of epitopes displayed on the surface of E. coli cells. Biol Chem 382, 1669-1677 (2001).
³⁸ Lu, Z. et al. Expression of Thioredoxin random peptide libraries on the Eschericia coli cell surface as functional fusions to flagellin: a system designed for exploring protein protein interactions. Bio/Technology 13, 366-372 (1995).
³⁹ Kiss, C. et al. In vitro evolution of protein thermostability by insertional destabilization, mutation and gene synthesis. Protein Eng Des Sel in press (2008).
⁴⁰ Manting, E. H. & Driessen, A. J. Escherichia coli translocase: the unravelling of a molecular machine. Mol Microbiol 37, 226-238 (2000).
⁴¹ de Gier, J. W. & Luirink, J. Biogenesis of inner membrane proteins in Escherichia coli. Mol Microbiol 40, 314-322 (2001).
⁴² Driessen, A. J., Manting, E. H. & van der Does, C. The structural basis of protein targeting and translocation in bacteria. Nat Struct Biol 8, 492-498 (2001).
⁴³ Muller, M., Koch, H. G., Beck, K. & Schafer, U. Protein traffic in bacteria: multiple routes from the ribosome to and across the membrane. Prog Nucleic Acid Res Mol Biol 66, 107-157 (2001).
⁴⁴ Koch, H. G., Moser, M. & Muller, M. Signal recognition particle-dependent protein targeting, universal to all kingdoms of life. Rev Physiol Biochem Pharmacol 146, 55-94 (2003).
⁴⁵ Mergulhao, F. J., Summers, D. K. & Monteiro, G. A. Recombinant protein secretion in Escherichia coli. Biotechnol Adv 23, 177-202 (2005).
⁴⁶ Berks, B. C. A common export pathway for proteins binding complex redox cofactors? Mol Microbiol 22, 393-404 (1996).
⁴⁷ Berks, B. C., Palmer, T. & Sargent, F. Protein targeting by the bacterial twin-arginine translocation (Tat) pathway. Curr Opin Microbiol 8, 174-181 (2005).
⁴⁸ Berks, B. C., Sargent, F. & Palmer, T. The Tat protein export pathway. Mol Microbiol 35, 260-274 (2000).
⁴⁹ Barrett, C. M., Ray, N., Thomas, J. D., Robinson, C. & Bolhuis, A. Quantitative export of a reporter protein, GFP, by the twin-arginine translocation pathway in Escherichia coli. Biochem Biophys Res Commun 304, 279-284 (2003).
⁵⁰ Ize, B., Gerard, F. & Wu, L. F. In vivo assessment of the Tat signal peptide specificity in Escherichia coli. Arch Microbiol 178, 548-553 (2002).
⁵¹ DeLisa, M. P., Samuelson, P., Palmer, T. & Georgiou, G. Genetic analysis of the twin arginine translocator secretion pathway in bacteria. J Biol Chem 277, 29825-29831 (2002).
⁵² Thomas, J. D., Daniel, R. A., Errington, J. & Robinson, C. Export of active green fluorescent protein to the periplasm by the twin-arginine translocase (Tat) pathway in Escherichia coli. Mol Microbiol 39, 47-53 (2001).
⁵³ Santini, C. L. et al. Translocation of jellyfish green fluorescent protein via the Tat system of Escherichia coli and change of its periplasmic localization in response to osmotic up-shock. J Biol Chem 276, 8159-8164 (2001).
⁵⁴ Fisher, A. C., Kim, W. & DeLisa, M. P. Genetic selection for protein solubility enabled by the folding quality control feature of the twin-arginine translocation pathway. Protein Sci 15, 449-458 (2006).
⁵⁵ Steiner, D., Forrer, P., Stumpp, M. T. & Pluckthun, A. Signal sequences directing cotranslational translocation expand the range of proteins amenable to phage display. Nat Biotechnol (2006).
⁵⁶ Paschke, M. & Hohne, W. A twin-arginine translocation (Tat)-mediated phage display system. Gene 350, 79-88 (2005).
⁵⁷ Velappan, N. et al. A comprehensive analysis of filamentous phage display vectors for cytoplasmic proteins. Nucleic Acid Res. submitted (2008).
⁵⁸ Fellouse, F. A. et al. Molecular recognition by a binary code. J Mol Biol 348, 1153-1162 (2005).
⁵⁹ Birtalan, S. et al. The Intrinsic Contributions of Tyrosine, Serine, Glycine and Arginine to the Affinity and Specificity of Antibodies. J Mol Biol 377, 1518-1528, doi:50022-2836(08)00169-1 [pii] 10.1016/j.jmb.2008.01.093 (2008).
⁶⁰ Fellouse, F. A., Barthelemy, P. A., Kelley, R. F. & Sidhu, S. S. Tyrosine plays a dominant functional role in the paratope of a synthetic antibody derived from a four amino acid code. J Mol Biol 357, 100-114 (2006).
⁶¹ Koide, A., Gilbreth, R. N., Esaki, K., Tereshko, V. & Koide, S. High-affinity single-domain binding proteins with a binary-code interface. Proc Natl Acad Sci U S A 104, 6632-6637 (2007).
⁶² Kiss, C. et al. Antibody binding loop insertions as diversity elements. Nucleic Acids Res 34, e132 (2006).
⁶³ Campbell, R. E. et al. A monomeric red fluorescent protein. Proc Natl Acad Sci U S A 99, 7877-7882 (2002).

What is claimed is:

1. A fluorobody comprising a fluorescent protein which is eCGP fluorescent protein or a derivative thereof, wherein heterologous polypeptides are inserted in at least two loop positions on the same face of the fluorescent protein.

2. The fluorobody according to claim 1, wherein the fluorescent protein is eCGP123, nsTGP(−1), nsTGP(−10) or nsTGP(−18).

3. The fluorobody according to claim 1, wherein the fluorescent protein is TGP.

4. The fluorobody according to claim 1, wherein the heterologous polypeptides are inserted in at least three loops.

5. The fluorobody according to claim 4, wherein the three loops are loop 2, loop 3 and loop 6.

6. The fluorobody according to claim 5, wherein the diversity in the heterologous polypeptides is limited to a restricted set set of amino acids.

7. A fluorescent protein having the amino acid sequence of SEQ ID NO: 3, SEQ ID NO: 6, SEQ ID NO: 8 or SEQ ID NO: 10.

8. A nucleic acid molecule comprising a polynucleotide encoding the fluorescent protein according to claim 7.

9. The nucleic acid molecule of claim 8, wherein the amino acid sequence of SEQ ID NO: 3 is encoded by the polynucleotide of SEQ ID NO: 4

10. The fluorobody according to claim 2, wherein the heterologous polypeptides are inserted in at least three loops.

11. The fluorobody according to claim 3, wherein the heterologous polypeptides are inserted in at least three loops.


SEQUENCE LISTING (34 sequences)

SEQ ID NO: 1 - protein, 225 aa, Artificial Sequence
  Synthetic protein encoded by DNA of SEQ ID NO 2
MSVIKPEMKIKLRMEGAVNGHKFVIEGEGIGKPYEGTQTLDLTVKEGAPLPFSYDILTPAFQYGNRAFTKYPKDIPDYFKQAFPEGYSWERSMTYEDQGICIATSDITMEGDCFFYKIRFDGTNFPPNGPVMQKKTLKWEPSTEKMYVRDGVLKGDVNMALLLEGGGHYRCDFKTTYKAKKDVRLPDAHEVDHRIEILSHDKDYNKVRLYEHAEARYSMLPSQAK

SEQ ID NO: 2 - DNA, 681 bp, Artificial Sequence
  Synthetic
atgtcagtaattaaaccggaaatgaaaattaaattgcgtatggaaggtgccgttaacggccataaatttgtaattgaaggagaaggaataggcaaaccatacgaaggaacccagaccctggatttaaccgtaaaagaaggcgcacctctccctttctcgtacgacatcctcaccccagccttccaatacggcaatcgcgctttcaccaaatacccaaaagatattccagactattttaaacaagcattccccgaaggctattcttgggaacgctctatgacctatgaagatcaaggaatttgtatcgctacctccgacattactatggaaggagactgttttttttataagattcgctttgatggaactaacttccccccgaacggccctgtaatgcaaaagaagaccttaaaatgggaacctagcaccgaaaaaatgtatgtacgcgacggagttcttaagggtgacgtaaacatggcacttctgctcgaaggaggtggacactaccgctgcgattttaaaaccacttataaagccaaaaaagatgttcgtcttccagatgcacacgaggtggaccaccgcattgaaatcctgagccacgataaagattataataaagttagactctatgaacacgccgaagcccgctattctatgttaccgtctcaggctaaagctagc

SEQ ID NO: 3 - protein, 225 aa, Artificial Sequence
  Synthetic protein encoded by DNA of SEQ ID NO4
MSVIKPEMKIKLRMEGAVNGHKFVIEGEGIGKPYEGTQTLDLTVKEGAPLPFSYDILTPAFQYGNRAFTKYPKDIPDYFKQAFPEGYSWERSMTYEDQGICIATSDITMEGDCFFYKIRFDGTNFPPNGPVMQKKTLKWEPSTEKMYVRDGVLKGDVNMALLLEGGGHYRCDFKTTYKAKKDVRLPDAHKVDHRIEILSHDKDYNKVRLYEHAEARYSMLPSQAK

SEQ ID NO: 4 - DNA, 689 bp, Artificial Sequence
  Synthetic
gcgcgcatgcctcagtaattaaaccggaaatgaaaattaaattgcgtatggaaggtgccgttaacggccataaatttgtaattgaaggagaaggaataggcaaaccatacgaaggaacccagaccctggatttaaccgtaaaagaaggcgcacctctccctttctcgtacgacatcctcaccccagccttccaatacggcaatcgcgctttcaccaaatacccaaaagatattccagactattttaaacaagcattccccgaaggctattcttgggaacgctctatgacctatgaagatcaaggaatttgtatcgctacctccgacattactatggaaggagactgttttttttataagattcgctttgatggaactaacttccccccgaacggccctgtaatgcaaaagaagaccttaaaatgggaacctagcaccgaaaaaatgtatgtacgcgacggagttcttaagggtgacgtaaacatggcacttctgctcgaaggaggtggacactaccgctgcgattttaaaaccacttataaagccaaaaaagatgttcgtcttccagatgcacacaaagtggaccaccgcattgaaatcctgagccacgataaagattataataaagttagactctatgaacacgccgaagcccgctattctatgttaccgtctcaggctaaagctagc

SEQ ID NO: 5 - DNA, 672 bp, Artificial Sequence
  Synthetic
tcagtaattaaaccggaaatgaaaattaaattgcgtatggaaggtgccgttaacggccataaatttgtaattgaaggagaaggaataggcaaaccatacgaaggaacccagaccctggatttaaccgtaaaagaaggcgcacctctccctttctcgtacgacatcctcaccccagccttccaatacggcaatcgcgctttcaccaaatacccaaaagatattccagactattttaaacaagcattccccgaaggctattcttgggaacgctctatgacctatgaagatcaaggaatttgtatcgctacctccgacattactatggaaggagactgttttttttataagattcgctttgatggaactaacttccccccgaacggccctgtaatgcaaaagaagaccttaaaatgggaacctagcaccgaaaaaatgtatgtacgcgacggagttcttaagggtgacgtaaacatggcacttctgctcgaaggaggtggacactaccgctgcgattttaaaaccacttataaagccaaaaaagatgttcgtcttccagatgcacacgaggtggaccaccgcattgaaatcctgagccacgataaagattataataaagttagactctatgaacacgccgaagcccgctattctggcggaggcagcggaggcggt

SEQ ID NO: 6 - protein, 227 aa, Artificial Sequence
  Synthetic protein encoded by DNA of SEQ ID NO 5
AHASVIKPEMKIKLRMEGAVNGHKFVIEGEGIGKPYEGTQTLDLTVKEGAPLPFSYDILTPAFQYGNRAFTKYPKDIPDYFKQAFPEGYSWERSMTYEDQGICIATSDITMEGDCFFYKIRFDGTNFPPNGPVMQKKTLKWEPSTEKMYVRDGVLKGDVNMALLLEGGGHYRCDFKTTYKAKKDVRLPDAHEVDHRIEILSHDKDYNKVRLYEHAEARYSGGGSGGG

SEQ ID NO: 7 - DNA, 672 bp, Artificial Sequence
  Synthetic
tcagtaattaaaccggaaatgaaaattaaattgcgtatggaaggtgccgttaacggccataaatttgtaattgaaggagaaggaataggcaaaccatacgaaggaacccagaccctggatttaaccgtagaagaaggcgcacctctccctttctcgtacgacatcctcaccccagccttccaatacggcaatcgcgctttcaccaaatacccagaagatattccagactattttaaacaagcattccccgaaggctattcttgggaacgctctatgacctatgaagatcaaggaatttgtatcgctacctccgacattactatggaaggagactgttttttttatgaaattcgctttgatggaactaacttccccccgaacggccctgtaatgcaaaagaagaccttaaaatgggaacctagcaccgaaaaaatgtatgtagaagacggagttcttaagggtgacgtagaaatggcacttctgctcgaaggaggtggacactaccgctgcgattttaaaaccacttataaagccaaaaaagatgttcgtcttccagatgcacacgaggtggaccaccgcattgaaatcctgagccacgataaagattataataaagttagactctatgaacacgccgaagcccgctattctggcggaggcagcggaggcggt

SEQ ID NO: 8 - protein, 224 aa, Artificial Sequence
  Synthetic protein encoded by DNA of SEQ ID NO 7
SVIKPEMKIKLRMEGAVNGHKFVIEGEGIGKPYEGTQTLDLTVEEGAPLPFSYDILTPAFQYGNRAFTKYPEDIPDYFKQAFPEGYSWERSMTYEDQGICIATSDITMEGDCFFYEIRFDGTNFPPNGPVMQKKTLKWEPSTEKMYVEDGVLKGDVEMALLLEGGGHYRCDFKTTYKAKKDVRLPDAHEVDHRIEILSHDKDYNKVRLYEHAEARYSGGGSGGG

SEQ ID NO: 9 - DNA, 672 bp, Artificial Sequence
  Synthetic
tcagtaattgaaccggaaatgaaaattaaattgcgtatggaaggtgccgttaacggccataaatttgtaattgaaggagaaggaataggcaaaccatacgaaggaacccagaccctggatttaaccgtagaagaaggcgcacctctccctttctcgtacgacatcctcaccccagccttccaatacggcaatcgcgctttcaccgaatacccagaagatattccagactattttaaacaagcattccccgaaggctattcttgggaacgctctatgacctatgaagatcaaggaatttgtatcgctacctccgacattactatggaaggagactgttttttttatgaaattgaatttgatggaactaacttccccccgaacggccctgtaatgcaaaagaagaccttaaaatgggaacctagcaccgaaaaaatgtatgtagaagacggagttcttaagggtgacgtagaaatggcacttctgctcgaaggaggtggacactaccgctgcgattttaaaaccacttataaagccaaaaaagatgttcgtcttccagatgcacacgaggtggaccacgaaattgaaatcctgagccacgataaagattataataaagttagactctatgaacacgccgaagcccgctattctggcggaggcagcggaggcggt

SEQ ID NO: 10 - protein, 224 aa, Artificial Sequence
  Synthetic protein encoded by DNA of SEQ ID NO 9
SVIEPEMKIKLRMEGAVNGHKFVIEGEGIGKPYEGTQTLDLTVEEGAPLPFSYDILTPAFQYGNRAFTEYPEDIPDYFKQAFPEGYSWERSMTYEDQGICIATSDITMEGDCFFYEIEFDGTNFPPNGPVMQKKTLKWEPSTEKMYVEDGVLKGDVEMALLLEGGGHYRCDFKTTYKAKKDVRLPDAHEVDHEIEILSHDKDYNKVRLYEHAEARYSGGGSGGG

SEQ ID NO: 11 - DNA, 50 bp, Artificial Sequence
  Synthetic primer oligonucleotide
gggaacgctctatgacctatkvykvykvyggaatttgtatcgctacctcc

SEQ ID NO: 12 - DNA, 53 bp, Artificial Sequence
  Synthetic primer oligonucleotide
gggaacgctctatgacctatkvykvykvykvyggaatttgtatcgctacctcc

SEQ ID NO: 13 - DNA, 56 bp, Artificial Sequence
  Synthetic primer oligonucleotide
gggaacgctctatgacctatkvykvykvykvykvyggaatttgtatcgctacctcc

SEQ ID NO: 14 - DNA, 59 bp, Artificial Sequence
  Synthetic primer oligonucleotide
gggaacgctctatgacctatkvykvykvykvykvykvyggaatttgtatcgctacctcc

SEQ ID NO: 15 - DNA, 62 bp, Artificial Sequence
  Synthetic primer oligonucleotide
gggaacgctctatgacctatkvykvykvykvykvykvykvyggaatttgtatcgctacctcc

SEQ ID NO: 16 - DNA, 65 bp, Artificial Sequence
  Synthetic primer oligonucleotide
gggaacgctctatgacctatkvykvykvykvykvykvykvykvyggaatttgtatcgctacctcc

SEQ ID NO: 17 - DNA, 68 bp, Artificial Sequence
  Synthetic primer oligonucleotide
gggaacgctctatgacctatkvykvykvykvykvykvykvykvykvyggaatttgtatcgctacctcc

SEQ ID NO: 18 - DNA, 71 bp, Artificial Sequence
  Synthetic primer oligonucleotide
gggaacgctctatgacctatkvykvykvykvykvykvykvykvykvykvyggaatttgtatcgctacctcc

SEQ ID NO: 19 - DNA, 74 bp, Artificial Sequence
  Synthetic primer oligonucleotide
gggaacgctctatgacctatkvykvykvykvykvykvykvykvykvykvykvyggaatttgtatcgctacctcc

SEQ ID NO: 20 - DNA, 77 bp, Artificial Sequence
  Synthetic primer oligonucleotide
gggaacgctctatgacctatkvykvykvykvykvykvykvykvykvykvykvykvyggaatttgtatcgctacctcc

SEQ ID NO: 21 - DNA, 80 bp, Artificial Sequence
  Synthetic primer oligonucleotide
gggaacgctctatgacctatkvykvykvykvykvykvykvykvykvykvykvykvykvyggaatttgtatcgctacctcc

SEQ ID NO: 22 - DNA, 64 bp, Artificial Sequence
  Synthetic primer oligonucleotide
gggtgacgtaaacatggcacttctgkvykvykvykvykvykvykvycgctgcgattttaaaacc

SEQ ID NO: 23 - DNA, 67 bp, Artificial Sequence
  Synthetic primer oligonucleotide
gggtgacgtaaacatggcacttctgkvykvykvykvykvykvykvykvycgctgcgattttaaaacc

SEQ ID NO: 24 - DNA, 70 bp, Artificial Sequence
  Synthetic primer oligonucleotide
gggtgacgtaaacatggcacttctgkvykvykvykvykvykvykvykvykvycgctgcgattttaaaacc

SEQ ID NO: 25 - DNA, 73 bp, Artificial Sequence
  Synthetic primer oligonucleotide
gggtgacgtaaacatggcacttctgkvykvykvykvykvykvykvykvykvykvycgctgcgattttaaaacc

SEQ ID NO: 26 - DNA, 76 bp, Artificial Sequence
  Synthetic primer oligonucleotide
gggtgacgtaaacatggcacttctgkvykvykvykvykvykvykvykvykvykvykvycgctgcgattttaaaacc

SEQ ID NO: 27 - DNA, 79 bp, Artificial Sequence
  Synthetic primer oligonucleotide
gggtgacgtaaacatggcacttctgkvykvykvykvykvykvykvykvykvykvykvykvycgctgcgattttaaaacc

SEQ ID NO: 28 - DNA, 82 bp, Artificial Sequence
  Synthetic primer oligonucleotide
gggtgacgtaaacatggcacttctgkvykvykvykvykvykvykvykvykvykvykvykvykvycgctgcgattttaaaacc

SEQ ID NO: 29 - DNA, 85 bp, Artificial Sequence
  Synthetic primer oligonucleotide
gggtgacgtaaacatggcacttctgkvykvykvykvykvykvykvykvykvykvykvykvykvykvycgctgcgattttaaaacc

SEQ ID NO: 30 - DNA, 75 bp, Artificial Sequence
  Synthetic primer oligonucleotide
gattcgctttgatggaactkvyttckvykvykvykvykvygtaatgkvyaagkvyaccttaaaatgggaacctag

SEQ ID NO: 31 - DNA, 75 bp, Artificial Sequence
  Synthetic primer oligonucleotide
gattcgctttgatggaactdvyttcbvbbvbdvykvybvbgtaatgbvbaagdvbaccttaaaatgggaacctag

SEQ ID NO: 32 - DNA, 75 bp, Artificial Sequence
  Synthetic primer oligonucleotide
gattcgctttgatggaactdvyttcbvbbvbdvyggcbvbgtaatgbvbaagdvbaccttaaaatgggaacctag

SEQ ID NO: 33 - DNA, 35 bp, Artificial Sequence
  Synthetic primer oligonucleotide
tacatatgggcgcgcatgcctcagtaattaaaccg

SEQ ID NO: 34 - DNA, 41 bp, Artificial Sequence
  Synthetic primer oligonucleotide
tttgccgctagctttagcctgagacggtaacatagaatagc